SEQUENCE LISTING (26 sequences)

SEQ ID NO: 1 - DNA, 1753 bp, Homo sapiens
cagccccgcccctacctgtggaagcccagccgcccgctcccgcggataaaaggcgcggagtgtccccgaggtcagcgagtgcgcgctcctcctcgcccgccgctaggtccatcccggcccagccaccatgtccatccacttcagctccccggtattcacctcgcgctcagccgccttctcgggccgcggcgcccaggtgcgcctgagctccgctcgccccggcggccttggcagcagcagcctctacggcctcggcgcctcacggccgcgcgtggccgtgcgctctgcctatgggggcccggtgggcgccggcatccgcgaggtcaccattaaccagagcctgctggccccgctgcggctggacgccgacccctccctccagcgggtgcgccaggaggagagcgagcagatcaagaccctcaacaacaagtttgcctccttcatcgacaaggtgcggtttctggagcagcagaacaagctgctggagaccaagtggacgctgctgcaggagcagaagtcggccaagagcagccgcctcccagacatctttgaggcccagattgctggccttcggggtcagcttgaggcactgcaggtggatgggggccgcctggaggcggagctgcggagcatgcaggatgtggtggaggacttcaagaataagtacgaagatgaaattaaccaccgcacagctgctgagaatgagtttgtggtgctgaagaaggatgtggatgctgcctacatgagcaaggtggagctggaggccaaggtggatgccctgaatgatgagatcaacttcctcaggaccctcaatgagacggagttgacagagctgcagtcccagatctccgacacatctgtggtgctgtccatggacaacagtcgctccctggacctggacggcatcatcgctgaggtcaaggcgcagtatgaggagatggccaaatgcagccgggctgaggctgaagcctggtaccagaccaagtttgagaccctccaggcccaggctgggaagcatggggacgacctccggaatacccggaatgagatttcagagatgaaccgggccatccagaggctgcaggctgagatcgacaacatcaagaaccagcgtgccaagttggaggccgccattgccgaggctgaggagcgtggggagctggcgctcaaggatgctcgtgccaagcaggaggagctggaagccgccctgcagcggggcaagcaggatatggcacggcagctgcgtgagtaccaggaactcatgagcgtgaagctggccctggacatcgagatcgccacctaccgcaagctgctggagggcgaggagagccggttggctggagatggagtgggagccgtgaatatctctgtgatgaattccactggtggcagtagcagtggcggtggcattgggctgaccctcgggggaaccatgggcagcaatgccctgagcttctccagcagtgcgggtcctgggctcctgaaggcttattccatccggaccgcatccgccagtcgcaggagtgcccgcgactgagccgcctcccaccactccactcctccagccaccacccacaatcacaagaagattcccacccctgcctcccatgcctggtcccaagacagtgagacagtctggaaagtgatgtcagaatagcttccaataaagcagcctcattctgaggcctgagtgatccacgtgaaaaaaaaaaaaaaaaaaaaaaaaaaaaaaaaaaaaaaaaaaaaaaaaaaa

SEQ ID NO: 2 - DNA, 1513 bp, Homo sapiens
cgcccctgacaccattcctcccttcccccctccaccggccgcgggcataaaaggcgccaggtgagggcctcgccgctcctcccgcgaatcgcagcttctgagaccagggttgctccgtccgtgctccgcctcgccatgacttcctacagctatcgccagtcgtcggccacgtcgtccttcggaggcctgggcggcggctccgtgcgttttgggccgggggtcgcctttcgcgcgcccagcattcacgggggctccggcggccgcggcgtatccgtgtcctccgcccgctttgtgtcctcgtcctcctcgggggcctacggcggcggctacggcggcgtcctgaccgcgtccgacgggctgctggcgggcaacgagaagctaaccatgcagaacctcaacgaccgcctggcctcctacctggacaaggtgcgcgccctggaggcggccaacggcgagctagaggtgaagatccgcgactggtaccagaagcaggggcctgggccctcccgcgactacagccactactacacgaccatccaggacctgcgggacaagattcttggtgccaccattgagaactccaggattgtcctgcagatcgacaatgcccgtctggctgcagatgacttccgaaccaagtttgagacggaacaggctctgcgcatgagcgtggaggccgacatcaacggcctgcgcagggtgctggatgagctgaccctggccaggaccgacctggagatgcagatcgaaggcctgaaggaagagctggcctacctgaagaagaaccatgaggaggaaatcagtacgctgaggggccaagtgggaggccaggtcagtgtggaggtggattccgctccgggcaccgatctcgccaagatcctgagtgacatgcgaagccaatatgaggtcatggccgagcagaaccggaaggatgctgaagcctggttcaccagccggactgaagaattgaaccgggaggtcgctggccacacggagcagctccagatgagcaggtccgaggttactgacctgcggcgcacccttcagggtcttgagattgagctgcagtcacagctgagcatgaaagctgccttggaagacacactggcagaaacggaggcgcgctttggagcccagctggcgcatatccaggcgctgatcagcggtattgaagcccagctgggcgatgtgcgagctgatagtgagcggcagaatcaggagtaccagcggctcatggacatcaagtcgcggctggagcaggagattgccacctaccgcagcctgctcgagggacaggaagatcactacaacaatttgtctgcctccaaggtcctctgaggcagcaggctctggggcttctgctgtcctttggagggtgtcttctgggtagagggatgggaaggaagggacccttacccccggctcttctcctgacctgccaataaaaatttatggtccaagggaaaaaaaaaaaaaaaaaaaaaaaaaaaaaaaaaaaaaaaaaaaaaaaaaa

SEQ ID NO: 3 - DNA, 503 bp, Homo sapiens
gacagcggcttccttgatccttgccacccgcgactgaacaccgacagcagcagcctcaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaactttctgcaagacctttggctcacagaactgcagggtatggtgagaaaccaactacggattgctgcaaaccacaccttctctttcttatgtctttttactacaaactacaagacaattgttgaaacctgctatacatgtttattttaataaattgatggca

SEQ ID NO: 4 - DNA, 517 bp, Homo sapiens
cctccacagcaacttccttgatccctgccacgcacgactgaacacagacagcagccgcctcgccatgaagctgctgatggtcctcatgctggcggccctcctcctgcactgctatgcagattctggctgcaaactcctggaggacatggttgaaaagaccatcaattccgacatatctatacctgaatacaaagagcttcttcaagagttcatagacagtgatgccgctgcagaggctatggggaaattcaagcagtgtttcctcaaccagtcacatagaactctgaaaaactttggactgatgatgcatacagtgtacgacagcatttggtgtaatatgaagagtaattaactttacccaaggcgtttggctcagagggctacagactatggccagaactcatctgttgattgctagaaaccacttttctttcttgtgttgtctttttatgtggaaactgctagacaactgttgaaacctcaaattcatttccatttcaataactaactgcaaatc

SEQ ID NO: 5 - DNA, 576 bp, Homo sapiens
cttctctgggacacattgccttctgttttctccagcatgcgcttgctccagctcctgttcagggccagccctgccaccctgctcctggttctctgcctgcagttgggggccaacaaagctcaggacaacactcggaagatcataataaagaattttgacattcccaagtcagtacgtccaaatgacgaagtcactgcagtgcttgcagttcaaacagaattgaaagaatgcatggtggttaaaacttacctcattagcagcatccctctacaaggtgcatttaactataagtatactgcctgcctatgtgacgacaatccaaaaaccttctactgggacttttacaccaacagaactgtgcaaattgcagccgtcgttgatgttattcgggaattaggcatctgccctgatgatgctgctgtaatccccatcaaaaacaaccggttttatactattgaaatcctaaaggtagaataatggaagccctgtctgtttgccacacccaggtgatttcctctaaagaaacttggctggaatttctgctgtggtctataaaataaacttcttaacatgctt

SEQ ID NO: 6 - DNA, 1528 bp, Homo sapiens
cggcgagcgagcaccttcgacgcggtccggggaccccctcgtcgctgtcctcccgacgcggacccgcgtgccccaggcctcgcgctgcccggccggctcctcgtgtcccactcccggcgcacgccctcccgcgagtcccgggcccctcccgcgcccctcttctcggcgcgcgcgcagcatggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcataactatataatttgaagattatagaagaagggaaatagcaaatggacacaaattacaaatgtgtgtgcgtgggacgaagacatctttgaaggtcatgagtttgttagtttaacatcatatatttgtaatagtgaaacctgtactcaaaatataagcagcttgaaactggctttaccaatcttgaaatttgaccacaagtgtcttatatatgcagatctaatgtaaaatccagaacttggactccatcgttaaaattatttatgtgtaacattcaaatgtgtgcattaaatatgcttccacagtaaaatctgaaaaactgatttgtgattgaaagctgcctttctatttacttgagtcttgtacatacatacttttttatgagctatgaaataaaacattttaaactg

SEQ ID NO: 7 - DNA, 20 bp, Artificial Sequence
  CK19 reverse PCR primer
agagcctgttccgtctcaaa

SEQ ID NO: 8 - DNA, 19 bp, Artificial Sequence
  CK7 reverse PCR primer
ccagggagcgactgttgtc

SEQ ID NO: 9 - DNA, 24 bp, Artificial Sequence
  MGB1 reverse PCR primer
ggaaatcacattctccaataaggg

SEQ ID NO: 10 - DNA, 24 bp, Artificial Sequence
  MGB1 probe
agccagagcctgcgtagcagtgct

SEQ ID NO: 11 - DNA, 25 bp, Artificial Sequence
  MGB2 reverse PCR Primer
ctgtcgtacactgtatgcatcatca

SEQ ID NO: 12 - DNA, 20 bp, Artificial Sequence
  PIP reverse PCR primer
gcagatgcctaattcccgaa

SEQ ID NO: 13 - DNA, 23 bp, Artificial Sequence
  TACSTD1 reverse PCR primer
ggttttgctcttctcccaagttt

SEQ ID NO: 14 - DNA, 19 bp, Artificial Sequence
  MGB1 RT primer
ggaaatcacattctccaat

SEQ ID NO: 15 - DNA, 17 bp, Artificial Sequence
  PIP RT primer
gcagatgcctaattccc

SEQ ID NO: 16 - DNA, 17 bp, Artificial Sequence
  TACSTD1 RT primer
agcccatcattgttctg

SEQ ID NO: 17 - DNA, 21 bp, Artificial Sequence
  PIP forward PCR primer
gcggctccagctcctgttcag

SEQ ID NO: 18 - DNA, 28 bp, Artificial Sequence
  PIP reverse PCR primer
ggcgcattatgatcttccgagtgttgtc

SEQ ID NO: 19 - DNA, 19 bp, Artificial Sequence
  PIP RT primer
gggaatgtcaaaattcttt

SEQ ID NO: 20 - DNA, 20 bp, Artificial Sequence
  TACSTD1 forward PCR primer
gcgcgttcgggcttctgctt

SEQ ID NO: 21 - DNA, 30 bp, Artificial Sequence
  TACSTD1 reverse PCR primer
gcgagttttcacagacacattcttcctgag

SEQ ID NO: 22 - DNA, 17 bp, Artificial Sequence
  TACSTD1 RT primer
agtttacggccagcttg

SEQ ID NO: 23 - DNA, 21 bp, Artificial Sequence
  GUS forward PCR primer
ctcatttggaattttgccgat

SEQ ID NO: 24 - DNA, 18 bp, Artificial Sequence
  GUS reverse PCR primer
ccgagtgaagatcccctt

SEQ ID NO: 25 - DNA, 26 bp, Artificial Sequence
  GUS probe
tgaacagtcaccgacgagagtgctgg

SEQ ID NO: 26 - DNA, 19 bp, Artificial Sequence
  GUS RT primer
tttgttgtctctgccgagt

(19) United States
(12) Patent Application Publication
(10) Pub. No.: US 2006/0068433 A1
(43) Pub. Date: Mar. 30, 2006

(54) Multiple mode multiplex reaction quenching method

(76) Inventors: Tony E. Godfrey, Bronxville, NY (US); William A. McMillan, Cupertino, CA (US)

Correspondence Address: JESSE A. HIRSHMAN, ESQ., 1722 MURRAY AVENUE, PITTSBURGH, PA, 15217, US

(21) Appl. No.: 11/230,412

(22) Filed: Sep. 20, 2005

Related U.S. Application Data
(60) Provisional application No. 60/611,400, filed on Sep. 20, 2004.

(51) Int. Cl. C12Q 1/68; C12P 19/34

(52) U.S. Cl. 435/6; 435/91.2

(57) ABSTRACT

Methods for balancing multiplexed PCR reactions are provided which exploit differences in primer and amplicon Tms. The methods may be controlled by a computer process. Also provided are articles of manufacture useful in such methods and compositions containing primers and probes useful in such methods.

### CROSS REFERENCE TO RELATED APPLICATIONS

Pursuant to 35 U.S.C. §119(e), this application claims priority to U.S. Provisional Patent Application No. 60/611,400, filed Sep. 20, 2004, which is incorporated herein by reference in its entirety.

### BACKGROUND

1. Field of the Invention

Provided are nucleic acid amplification methods and compositions and processes for implementing those methods.

2. Description of the Related Art

Expression levels of messenger RNA (mRNA) species can be quantified by a number of methods. Traditional methods include Northern blot analysis. Superior nucleic acid detection methods have been devised that facilitate quantification of transcripts. These methods involve amplification of the mRNA species by one of many nucleic amplification methods, such as quantitative reverse transcriptase polymerase chain reaction (QRT-PCR) methods, as are broadly known. Examples of useful PCR methods are described in U.S. patent application Ser. No. 10/090,326 (U.S. Ser. No. 10/090,326, US Patent Publication No. 20030017482), incorporated herein by reference in its entirety. Other nucleic acid amplification methods for determining expression levels of a given mRNA include isothermic amplification or detection assays and array technologies.

A typical PCR reaction includes multiple amplification steps, or cycles that selectively amplify a target nucleic acid species. Because detection of mRNA is necessary, the PCR reaction is coupled with a reverse transcription step (reverse transcription PCR, or RT-PCR). A typical PCR reaction includes three steps: a denaturing step in which a target nucleic acid is denatured; an annealing step in which a set of PCR primers (forward and backward primers) anneal to complementary DNA strands; and an elongation step in which a thermostable DNA polymerase elongates the primers. By repeating this step multiple times, a DNA fragment is amplified to produce an amplicon, corresponding to the target DNA sequence. Typical PCR reactions include 30 or more cycles of denaturation, annealing and elongation. In many cases, the annealing and elongation steps can be performed concurrently, that is at the same temperature, in which case the cycle contains only two steps. The lengths of the denaturation, annealing and elongation stages may be any effective length of time. Reverse transcriptase PCR, as well as other nucleic amplification methods for quantifying levels of an RNA target in a sample, typically include a reverse transcriptase reaction to convert the RNA target to an amplifiable cDNA target.

The relative prevalence of any given target nucleic acid in a sample typically is proportional to the speed of accumulation of the amplicon product in an amplification reaction. By speed of accumulation of a given amplicon, it is meant the time it takes to reach a threshold level of target-specific amplicons in a reaction, or in the case of a cycling method, such as PCR, the number of amplification cycles it takes to reach a threshold level (Ct). Accumulation of a target-specific amplicon may be measured by any method known in the art, but typically is detected by accumulation, or sometimes depletion, of a target fluorochrome. The ubiquitous TaqMan assay (fluorescent 5′ nuclease assay) as well as molecular beacon and scorpion technologies may be used to detect accumulation of target-specific amplicons.

As a non-limiting example of a typical nucleic acid amplification method, the fluorescent 5′ nuclease assay may be conducted on a single target nucleic acid species, but more typically is multiplexed. That is, two or more target DNA or mRNA sequences (species) are amplified in a single tube, for example, a target mRNA species and an endogenous control. In multiplex methods, care must be taken to balance the two or more amplification reactions. Prevalence of one target sequence as compared to other target sequences typically affects the relative accumulation of the two or more amplicons, skewing the results of the multiplexed reaction as a result of the limited resources present in the single reaction tube, such resources including nucleotides and polymerase enzyme.

As described in U.S. Ser. No. 10/090,326, multiplexed PCR assays may be optimized, or balanced, by time-shifting the production of amplicons or by manipulating primer Tms. Also it is well-known to manipulate primer concentrations to balance multiplex PCR reaction. Although effective in many instances, additional tools useful in balancing multiplex amplification reactions are welcome.

### SUMMARY

A multiplexed nucleic acid amplification method is provided. The method comprises exploiting differences in amplicon Tm to balance a multiplexed amplification. More specifically, the method comprises amplifying in a reaction mix a first nucleic acid sequence to produce a first amplicon and a second nucleic acid to produce a second amplicon, the first amplicon having a first Tm and the second amplicon having a second Tm that is different from the first Tm. The reaction comprises two stages comprising two protocols with different amplicon denaturation conditions, such that relative accumulation of the first amplicon and the second amplicon is modulated. In one embodiment, the method comprises monitoring accumulation of the first and the second amplicons in the reaction mix. In a further embodiment, a first fluorescent indicator accumulates in the reaction mix as a result of the accumulation of the first amplicon and a second, different fluorescent indicator accumulation in the reaction mix as a result of the accumulation of the second amplicon, such as in, without limitation, a fluorescent 5′ endonuclease (TAQMAN) assay. In a further embodiment, the reaction mix comprises a first primer set and a second primer set having different Tms and wherein the reaction contains two stages with different primer annealing conditions so that relative accumulation of amplicons produced by the first primer set and the second primer set is different between the two stages.

In another embodiment, the amplifying is conducted in thermal cycler in which cycle reaction protocols are controlled by a computer device according to the following steps: monitoring one or more reporters in a stage, wherein accumulation of the one or more reporters corresponds to accumulation of one or more amplicons in the reaction mixture; and advancing to a next reaction stage when accumulation of the one or more reporters crosses a threshold level. In various further embodiments, reaction protocols may be changed when the reaction is advanced to a next stage; one or more additional cycles may be conducted after threshold crossing, but before advancing to a new stage; the reaction may be terminated if none of the monitored reporters cross the threshold; the reaction may be terminated a fixed number of cycles after advancing to next stage; and/or the threshold crossings of different reporters may advance to either the same or different stages.

In a further embodiment, a multiplexed method of identifying expression of markers indicative of the presence of breast cancer cells in a lymph node of a patient is provided, substantially as described herein. In one embodiment, the method comprises a multiplexed QRT-PCR reaction in which TACSTD1, PIP and an endogenous control mRNA expression levels in lymph nodes are quantified. One or more primers or probes listed in tables B, C and I, below, or derivatives or analogs thereof may be used in the multiplexed method.

### BRIEF DESCRIPTION OF THE DRAWINGS

FIG. 1 is a listing of a cDNA sequence of the cytokeratin 7 (CK7) marker (SEQ ID NO: 1).

FIG. 2 is a listing of a cDNA sequence of the cytokeratin 19 (CK19) marker (SEQ ID NO: 2).

FIG. 3 is a listing of a cDNA sequence of the mammaglobin 1 (MGB1) marker (SEQ ID NO: 3).

FIG. 4 is a listing of a cDNA sequence of the mammaglobin 2 (MGB2) marker (SEQ ID NO: 4).

FIG. 5 is a listing of a cDNA sequence of the prolactin-inducible protein (PIP) marker (SEQ ID NO: 5).

FIG. 6 is a listing of a cDNA sequence of the tumor-associated calcium signal transducer 1 (TACSTD1) marker (SEQ ID NO: 6).

FIG. 7 is a scatter plot showing the expression levels of CK7, CK19, MGB1, MGB2, PIP and TACSTD1 in primary tumor, tumor-positive lymph nodes and benign lymph nodes of a breast cancer patient (CK7—diamond; CK19—square; MGB1—light triangle; MGB2—empty triangle; PIP—asterix; and TACSTD1—bar).

FIGS. 8A-O provide scatter plots illustrating the ability of two-marker systems to distinguish between benign and malignant cells in a lymph node of a breast cancer patient (negative—light gray circle; positive—dark gray circle).

FIG. 9A provides data obtained from the secondary screening set of lymph nodes on individual gene expression observed in primary tumors, benign and positive nodes. The horizontal line indicates the most accurate cutoff value calculated by a receiver-operator characteristic curve analysis. Classification characteristics of the individual markers are reported in Table G, below.

FIGS. 9B-E provide secondary screening set data on gene expression for potential two-marker combinations using a linear discriminator decision rule. As with the individual markers, the black line indicates the decision rule generated from the secondary screening set data that produces the most accurate characterization. Classification characteristics of the marker combinations are reported in Table G.

FIGS. 9F-I provide secondary screening set data on gene expression for potential two-marker combinations applying equal probability contour statistical analysis. Equal probability curves were generated around the mean expression value observed for the 2 markers in benign lymph nodes. This demonstrates that while the marker combination of CK19 and MGB1 accurately characterizes the lymph nodes (see Table G), the wide distribution of expression observed in benign nodes for these markers increases optimism in applying the decision rule. By this method of analysis, the marker combination of TACSTD1 and PIP more confidently characterizes the lymph nodes.

FIG. 10A provides data obtained from the validation set of SLN on individual gene expression observed in negative and positive nodes. The horizontal line indicates the decision rule calculated from data obtained from the secondary screening set. Classification characteristics of the individual markers are reported in Table H.

FIGS. 10B-G provide validation set data on gene expression for two-marker combinations using linear discriminator decision rule for all potential marker pairs. As with the individual markers, the black line indicates the decision rule generated from the secondary screening set data that produces the most accurate characterization. Classification characteristics of the marker combinations are reported in Table H.

FIGS. 10H-M provide validation set data analyzed using the equal probability contours generated from the secondary screening set data. The relative levels of expression observed for all but one of the positive lymph nodes were well outside the 0.999 confidence contour. Some of the positive nodes are positive for only one marker (red crosses located in the left upper or right lower quadrants), demonstrating that a 2 marker assay improves sensitivity while maintaining high specificity.

FIGS. 11A-B provides results of a fully automated, 2-marker QRT-PCR analysis of lymph nodes. By either linear decision rule analysis (FIG. 11A) or equal probability contour analysis (FIG. 11B), the assay accurately characterized all 18 lymph nodes (9 negative, 9 positive) evaluated.

FIG. 12 is a graph depicting the effect of anneal temperature on performance of β-GUS primers.

FIG. 13 is a graph depicting the effect of anneal temperature on PIP PCR performance.

FIG. 14 is a graph depicting the effect of anneal temperature on TACSTD1 PCR performance.

FIG. 15 is a graph depicting the effect of temperature on target performance.

FIG. 16 is a graph depicting the linear dynamic range of the β-GUS, PIP, TACSTD1 triplex assay as described in Example 4.

FIG. 17 is a graph depicting the MDA-MB-453 total RNA test on GeneXpert® with “advance to next stage” turned off.

FIG. 18 is a graph depicting the MDA-MB-453 total RNA test on GeneXpert® with “advance to next stage” turned on.

FIG. 19 is a graph depicting the Human lymph node total RNA test on GeneXpert® with “advance to next stage” turned off.

FIG. 20 is a graph depicting the Human lymph node total RNA test on GeneXpert® with “advance to next stage” turned on.

FIG. 21 illustrates one embodiment of a system for implementing one embodiment of the ANS processes.

FIG. 22 is a flow diagram of one embodiment of an ANS process.

### DETAILED DESCRIPTION

Provided are methods and compositions useful in balancing multiplexed nucleic acid amplification reactions, such as PCR reactions and RT-PCR reactions, that involve a denaturing and primer annealing step. The method involves exploiting differential Tms of amplicons and primers to quench more favorable amplifications, thereby permitting less-favorable reactions to proceed without, or with less, competition for reaction resources identifying breast cancer and lung cancer cells, including micrometastases, in lymph nodes. Early detection of metastases typically is related to patient survival. Very small metastases often go undetected in histological study of lymph node biopsies, resulting in false negative results that result in decreased chances of patient survival. The nucleic acid detection assays described herein are much more discriminating than are histological studies in most instances (a few, excellent histologists are capable of identifying micrometastases in lymph node sections), and are robust and repeatable in the hands of any minimally-trained technician. Although the methods and compositions described herein are necessarily presented comprising expression of specific mRNA markers, it should be understood that it shall not be deemed to exclude methods and compositions comprising combinations of the specific markers and other markers known in the art. To this end, in one non-limiting embodiment, a number of molecular markers are identified that are expressed in breast cancer. These markers are markers specific to the tissue from which the particular cancer type arises and typically are not expressed, at least to the same levels, in lymphoid tissue. The presence and/or elevated expression of one or more of these markers in sentinel lymph node tissue is indicative of displaced cells in the lymphoid tissue, which correlates strongly with a cancer diagnosis.

As used herein, the terms “expression” and “expressed” mean production of a gene-specific mRNA by a cell. In the context of the present disclosure, a “marker” is a gene that is expressed abnormally in a lymphatic biopsy. In one embodiment, the markers described herein are mRNA species that are expressed in cells of a specific tumor source at a significantly higher level as compared to expression in lymphoid cells.

The improved PCR methods described herein, as well as those described in U.S. Ser. No. 10/090,326, permit rapid detection of cancer cells in lymph node tissue. These rapid methods can be used intraoperatively, and also are useful in detecting rare nucleic acid species, even in multiplexed PCR reactions that concurrently detect a more prevalent control nucleic acid.

As described above, a typical PCR reaction includes multiple amplification steps, or cycles that selectively amplify a target nucleic acid species. Because detection of transcripts is necessary, the PCR reaction is coupled with a reverse transcription step. A typical PCR reaction includes three steps: a denaturing step in which a target nucleic acid is denatured; an annealing step in which a set of PCR primers (forward and backward primers) anneal to complementary DNA strands; and an elongation step in which a thermostable DNA polymerase elongates the primers. By repeating this step multiple times, a DNA fragment is amplified to produce an amplicon, corresponding to the target DNA sequence. Typical PCR reactions include 30 or more cycles of denaturation, annealing and elongation. In many cases, the annealing and elongation steps can be performed concurrently, that is at the same temperature, in which case the cycle contains only two steps.

The lengths of the denaturation, annealing and elongation stages may be any desirable length of time. However, in attempting to shorten the PCR amplification reaction to a time suitable for intraoperative diagnosis, the lengths of these steps can be in the seconds range, rather than the minutes range. The denaturation step may be conducted for times of one second or less. The annealing and elongation steps optimally are less than 10 seconds each, and when conducted at the same temperature, the combination annealing/elongation step may be less than 10 seconds. Use of recently-developed amplification techniques, such as conducting the PCR reaction in a Rayleigh-Benard convection cell, also can dramatically shorten the PCR reaction time beyond these time limits (see, Krishnan, My et al., “PCR in a Rayleigh-Benard convection cell.” Science 298:793 (2002), and Braun, D. et al., “Exponential DNA Replication by Laminar Convection,” Physical Review Letters, 91:158103).

As described in U.S. Ser. No. 10/090,326, each cycle may be shortened considerably without substantial deterioration of production of amplicons. Use of high concentrations of primers is helpful in shortening the PCR cycle time. High concentrations typically are greater than about 400 nM, and often greater than about 800 nM, though the optimal concentration of primers will vary somewhat from assay-to-assay. Sensitivity of RT-PCR assays may be enhanced by the use of a sensitive reverse transcriptase enzyme (described below) and/or high concentrations of reverse transcriptase primer to produce the initial target PCR template. The specificity of any given PCR reaction relies heavily, but not exclusively, on the identity of the primer sets. The primer sets are pairs of forward and reverse oligonucleotide primers that anneal to a target DNA sequence to permit amplification of the target sequence, thereby producing a target sequence-specific amplicon. PCR primer sets can include two primers internal to the target sequence, or one primer internal to the target sequence and one specific to a target sequence that is ligated to the DNA or cDNA target, using a technique known as “ligation-anchored PCR” (Troutt, A. B., et al. (1992), “Ligation-anchored PCR: A Simple Amplification Technique with Single-sided Specificity,” Proc. Natl. Acad. Sci. USA, 89:98239825).

As used herein, a “derivative” of a specified oligonucleotide is an oligonucleotide that binds to the same target sequence as the specified oligonucleotide and amplifies the same target sequence to produce essentially the same amplicon as the specified oligonucleotide but for differences between the specified oligonucleotide and its derivative. The derivative may differ from the specified oligonucleotide by insertion, deletion and/or substitution of any residue of the specified sequence so long as the derivative substantially retains the characteristics of the specified sequence in its use for the same purpose as the specified sequence. By “analog”, it is meant an RNA or DNA equivalent having one or more modified bases and/or a modified backbone, including, for example and without limitation, phosphorothioate nucleic acids and peptide nucleic acids.

As used herein, in the context of gene expression analysis, a probe is “specific to” a gene or transcript if under reaction conditions it can hybridize specifically to transcripts of that gene within a sample, or sequences complementary thereto, and not to other transcripts. Thus, in a diagnostic assay, a probe or primer is “specific to” a gene if it can bind to a specific transcript or desired family of transcripts in mRNA extracted from a specimen, to the practical exclusion (does not interfere substantially with the detection assay) of other transcripts. In a PCR assay, primers are specific to a gene if they specifically amplify a sequence of that gene, to the practical exclusion of other sequences in a sample.

As used herein, a “primer or probe” for detecting a specific mRNA species is any primer, primer set and/or probe that can be utilized to detect and/or quantify the specific mRNA species. An “mRNA species” can be a single mRNA species, corresponding to a single mRNA expression product of a single gene, or can be multiple mRNAs that are detected by a single common primer and/or probe combination.

As used herein, “reagents” for any assay or reaction, such as a reverse transcription and PCR, are any compound or composition that is added to the reaction mixture including, without limitation, enzyme(s), nucleotides or analogs thereof, primers and primer sets, probes, antibodies or other binding reagents, detectable labels or tags, buffers, salts and co-factors. As used herein, unless expressed otherwise, a “reaction mixture” or “reaction mix” for a given assay or reaction includes combinations of compound(s) and/or composition(s) useful in performing that assay or reaction, even if those compounds or compositions are not expressly indicated. Reagents for many common assays or reactions, such as enzymatic reaction, are known in the art and typically are provided and/or suggested when the assay or reaction kit is sold.

As also described in U.S. Ser. No. 10/090,326, multiplexed PCR assays may be optimized, or balanced, by time-shifting the production of amplicons, rather than by manipulating primer concentrations. This may be achieved by using two primer sets, each primer set having a different Tm so that a two-stage PCR assay can be performed, with different annealing and/or elongation temperatures for each stage to favor the production of one amplicon over another. This time and temperature shifting method permits optimal balancing of the multiplex reaction without the difficulties faced when manipulation of primer concentrations is used to balance the reaction. This technique is especially useful in a multiplex reaction where it is desirable to amplify a rare cDNA along with a control cDNA.

A quantitative reverse transcriptase polymerase chain reaction for rapidly and accurately detecting low abundance RNA species in a population of RNA molecules (for example, and without limitation, total RNA or mRNA), includes the steps of: a) incubating an RNA sample with a reverse transcriptase and a reverse transcriptase primer, for example and without limitation, with a high concentration of a target sequence-specific reverse transcriptase primer and/or random oligonucleotides under conditions suitable to generate cDNA; b) subsequently adding suitable polymerase chain reaction (PCR) reagents to the reverse transcriptase reaction, including, for example, a high concentration of a PCR primer set specific to the cDNA and a thermostable DNA polymerase to the reverse transcriptase reaction, and c) cycling the PCR reaction for a desired number of cycles and under suitable conditions to generate PCR product (“amplicons”) specific to the cDNA. By temporally separating the reverse transcriptase and the PCR reactions, and by using reverse transcriptase-optimized and PCR-optimized primers, excellent specificity is obtained. The reaction may be conducted in a single tube (all tubes, containers, vials, cells and the like in which a reaction is performed may be referred to herein, from time to time, generically, as a “reaction vessel”), removing a source of contamination typically found in two-tube reactions. These reaction conditions permit very rapid QRT-PCR reactions, typically on the order of 20 minutes from the beginning of the reverse transcriptase reaction to the end of a 40 cycle PCR reaction.

The reaction c) may be performed in the same tube as the reverse transcriptase reaction by adding sufficient reagents to the reverse transcriptase (RT) reaction to create good, or even optimal conditions for the PCR reaction to proceed. A single tube may be loaded, prior to the running of the reverse transcriptase reaction, with: 1) the reverse transcriptase reaction mixture, and 2) the PCR reaction mixture to be mixed with the cDNA mixture after the reverse transcriptase reaction is completed. The reverse transcriptase reaction mixture and the PCR reaction mixture may be physically separated by a solid, or semi-solid (including amorphous, glassy substances and waxy) barrier of a composition that melts at a temperature greater than the incubation temperature of the reverse transcriptase reaction, but below the denaturing temperature of the PCR reaction. The barrier composition may be hydrophobic in nature and forms a second phase with the RT and PCR reaction mixtures when in liquid form. One example of such a barrier composition is wax beads, commonly used in PCR reactions, such as the AMPLIWAX PCR GEM products commercially available from Applied Biosystems of Foster City, Calif.

Alternatively, the separation of the reverse transcriptase and the PCR reactions may be achieved by adding the PCR reagents, including the PCR primer set and thermostable DNA polymerase, after the reverse transcriptase reaction is completed. Preferably the PCR reagents are added mechanically by a robotic or fluidic means to make sample contamination less likely and to remove human error.

The products of a QRT-PCR process, including multiplexed methods, may be compared after a fixed number of PCR cycles to determine the relative quantity of the RNA species as compared to a given reporter gene, such as, without limitation, an endogenous control or internal positive control, as described herein. One method of comparing the relative quantities of the products of the QRT-PCR process is by gel electrophoresis, for instance, by running the samples on a gel and detecting those samples by one of a number of known methods including, without limitation, Southern blotting and subsequent detection with a labeled probe, staining with ethidium bromide and incorporating fluorescent or radioactive tags in the amplicons.

However, the progress of the quantitative PCR reactions typically is monitored by determining the relative rates of amplicon production for each PCR primer set. Monitoring amplicon production may be achieved by a number of processes, including without limitation, fluorescent primers, fluorogenic probes and fluorescent dyes that bind double-stranded DNA.

A common method of measuring accumulation of an amplicon is the fluorescent 5′ nuclease assay. This method exploits the 5′ nuclease activity of certain thermostable DNA polymerases (such as Taq or Tfl DNA polymerases) to cleave an oligomeric probe during the PCR process. The oligomer is selected to anneal to the amplified target sequence under elongation conditions. The probe typically has a fluorescent reporter on its 5′ end and a fluorescent quencher of the reporter at the 3′ end. So long as the oligomer is intact, the fluorescent signal from the reporter is quenched. However, when the oligomer is digested during the elongation process, the fluorescent reporter no longer is in proximity to the quencher. The relative accumulation of free fluorescent reporter for a given amplicon may be compared to the accumulation of the same amplicons for a control sample and/or to that of a endogenous control gene, such as β-actin or 18S rRNA to determine the relative abundance of a given cDNA product of a given RNA in a RNA population. Products and reagents for the fluorescent 5′ nuclease assay are readily available commercially, for instance from Applied Biosystems.

For multiplex monitoring of the fluorescent 5′ nuclease assay, oligomers are provided corresponding to each amplicon species to be detected. The oligomer probe for each amplicon species has a fluorescent reporter with a different peak emission wavelength than the oligomer probe(s) for each other amplicons species. The accumulation of each unquenched fluorescent reporter can be monitored to determine the relative amounts of the target sequence corresponding to each amplicon.

Equipment and software also are readily available for monitoring amplicon accumulation in PCR and QRT-PCR according to the fluorescent 5′ nuclease assay and other QPCR/QRT-PCR procedures, including the SmartCycler®, commercially available from Cepheid of Sunnyvale, Calif. and the ABI Prism 7700 or 7900HT products (TaqMan), commercially available from Applied Biosystems. A cartridge-based sample preparation system (GeneXpert®, commercially available from Cepheid) combines a thermal cycler and fluorescent detection device having the capabilities of the SmartCycler® product with fluid circuits and processing elements capable of automatically extracting specific nucleic acids from a tissue sample and performing QPCR or QRT-PCR on the nucleic acid. The system uses disposable cartridges that can be configured and pre-loaded with a broad variety of reagents. Such a system can be configured to disrupt tissue and extract total RNA or mRNA from the sample. The reverse transcriptase reaction components can be added automatically to the RNA and the QPCR reaction components can be added automatically upon completion of the reverse transcriptase reaction.

Further, the PCR reaction may be monitored for production (or loss) of a particular fluorochrome from the reaction. When the fluorochrome levels reach (or fall to) a desired level, the automated system will automatically alter the PCR conditions. In one example, this is particularly useful in the multiplexed embodiment described above, where a more-abundant (control) target species is amplified by the first, lower Tm, primer set at a lower temperature than the less abundant species amplified by the second, higher Tm, primer set. In the first stage of the PCR amplification, the annealing temperature is lower than the effective Tm of the first primer set. The annealing temperature then is automatically raised above the effective Tm of the first primer set when production of the first amplicon by the first primer set is detected. In a system that automatically dispenses multiple reagents from a cartridge, such as the GeneXpert® system, a first PCR reaction may be conducted at the first Tm and, when the first PCR reaction proceeds past a threshold level, a second primer with a different Tm is added, resulting in a sequential multiplexed reaction.

In the above-described reactions, the amounts of certain reverse transcriptase and the PCR reaction components typically are atypical in order to take advantage of the faster ramp times of some thermal cyclers. Specifically, the primer concentrations are very high. Typical gene-specific primer concentrations for reverse transcriptase reactions are less than about 20 nM. To achieve a rapid reverse transcriptase reaction on the order of one to two minutes, the reverse transcriptase primer concentration was raised to greater than 20 nM, preferably at least about 50 nM, and typically about 100 nM. Standard PCR primer concentrations range from 100 nM to 300 nM. Higher concentrations may be used in standard PCR reactions to compensate for Tm variations. However, the referenced primer concentrations are for circumstances where no Tm compensation is needed. Proportionately higher concentrations of primers may be empirically determined and used if Tm compensation is necessary or desired. To achieve rapid PCR reactions, the PCR primer concentrations typically are greater than 200 nM, preferably greater than about 500 nM and typically about 800 nM. Typically, the ratio of reverse transcriptase primer to PCR primer is about 1 to 8 or more. The increase in primer concentrations permitted PCR experiments of 40 cycles to be conducted in less than 20 minutes.

A sensitive reverse transcriptase may be preferred in certain circumstances where either low amounts of RNA are present or a target RNA is a low abundance RNA. By the term “sensitive reverse transcriptase,” it is meant a reverse transcriptase capable of producing suitable PCR templates from low copy number transcripts for use as PCR templates. The sensitivity of the sensitive reverse transcriptase may derive from the physical nature of the enzyme, or from specific reaction conditions of the reverse transcriptase reaction mixture that produces the enhanced sensitivity. One example of a sensitive reverse transcriptase is SensiScript RT reverse transcriptase, commercially available from Qiagen, Inc. of Valencia, Calif. This reverse transcriptase is optimized for the production of cDNA from RNA samples of <50 ng, but also has the ability to produce PCR templates from low copy number transcripts. In practice, in the assays described herein, adequate results were obtained for samples of up to, and even in excess of, about 400 ng RNA. Other sensitive reverse transcriptases having substantially similar ability to reverse transcribe low copy number transcripts would be equivalent sensitive reverse transcriptase for the purposes described herein. Notwithstanding the above, the ability of the sensitive reverse transcriptase to produce cDNA from low quantities of RNA is secondary to the ability of the enzyme, or enzyme reaction system to produce PCR templates from low copy number sequences.

As discussed above, the procedures described herein also may be used in multiplex QRT-PCR processes. In its broadest sense, a multiplex PCR process involves production of two or more amplicons in the same reaction vessel. Typically, a multiplexed PCR assay produces amplicons specific to two different mRNA species to quantify relative amounts of the two mRNA species in an RNA sample. Nevertheless, a multiplexed assay can amplify two different DNA targets, and preferably one primer set does not amplify a target sequence within an amplicon produced by another primer set in the multiplexed reaction—that is, the two amplification reactions are not “nested.” Multiplex amplicons may be analyzed by methods described above.

In traditional multiplex QPCR and QRT-PCR procedures, the selection of PCR primer sets having similar annealing and elongation kinetics and similar sized amplicons are desirable. The design and selection of appropriate PCR primer sets is a process that is well known to a person skilled in the art. The process for identifying optimal PCR primer sets, and respective molar ratios thereof to achieve a balanced multiplex reaction also is known. By “balanced,” it is meant that certain amplicon(s) do not out-compete the other amplicon(s) for resources, such as dNTPs or enzyme. For instance, by limiting the abundance of the PCR primers for the more abundant RNA species in an RT-PCR experiment will allow the detection of less abundant species. Equalization of the Tm (melting temperature) for all PCR primer sets also is encouraged. See, for instance, ABI Prism 7700 Sequence Detection System User Bulletin #5, “Multiplex PCR with TaqMan VIC Probes”, Applied Biosystems (1998/2001).

Despite the above, for very low copy number transcripts, it is difficult to design accurate multiplex PCR experiments, even by limiting the PCR primer sets for the more abundant control species. One solution to this problem is to run the PCR reaction for the low abundance RNA in a separate tube than the PCR reaction for the more abundant species. However, that strategy does not take advantage of the benefits of running a multiplex PCR experiment. A two-tube process has several drawbacks, including cost, the addition of more room for experimental error and the increased chance of sample contamination.

A method has been described in U.S. Ser. No. 10/090,326 for performing a multiplex PCR process, including QRT-PCR and QPCR, capable of detecting low copy number nucleic acid species along with one or more higher copy number species. The difference between low copy number and high copy number nucleic acid species is relative, but is referred to herein as a difference in the prevalence of a low (lower) copy number species and a high (higher) copy number species of at least about 30-fold, but more typically at least about 100-fold. For purposes herein, the relative prevalence of two nucleic acid species to be amplified is more salient than the relative prevalence of the two nucleic acid species in relation to other nucleic acid species in a given nucleic acid sample because other nucleic acid species in the nucleic acid sample do not directly compete with the species to be amplified for PCR resources.

As used herein, the prevalence of any given nucleic acid species in a given nucleic acid sample, prior to testing, is unknown. Thus, the “expected” number of copies of a given nucleic acid species in a nucleic acid sample often is used herein and is based on historical data on the prevalence of that species in nucleic acid samples. For any given pair of nucleic acid species, one would expect, based on previous determinations of the relative prevalence of the two species in a sample, the prevalence of each species to fall within an expected range. By determining these ranges one would determine the -fold difference in the expected number of target sequences for each species.

The multiplex method involves performing a two- (or more) stage PCR amplification, permitting modulation of the relative rate of production of a first amplicon by a first primer set and a second amplicon by a second primer set during the respective amplification stages. By this method, PCR amplifications to produce amplicons directed to a lower abundance nucleic acid species are effectively “balanced” with PCR amplifications to produce amplicons directed to a higher abundance nucleic acid species. Separating the reaction into two or more temporal stages may be achieved by omitting the PCR primer set for any amplicons that are not to be produced in the first amplification stage. This is best achieved through use of automated processes, such as the GeneXpert® system described above. Two or more separate amplification stages may be used to tailor and balance multiplex assays, along with, or to the exclusion of tailoring the relative concentrations of the respective primer sets.

A second method for temporally separating the PCR amplification process into two or more stages is to select PCR primer sets with variation in their respective Tm. In one example, primers for a lower copy number nucleic acid species would have a higher Tm (Tm₁) than primers for a higher abundance species (Tm₂). In this process, the first stage of PCR amplification is conducted for a predetermined number of cycles at a temperature sufficiently higher than Tm₂ so that there is substantially no amplification of the higher abundance species. After the first stage of amplification, the annealing and elongation steps of the PCR reaction are conducted at a lower temperature, typically about Tm₂, so that both the lower abundance and the higher abundance amplimers are amplified. It should be noted that Tm, as used herein and unless otherwise noted, refers to “effective Tm,” which is the Tm for any given primer in a given reaction mix, which depends on factors, including, without limitation, the nucleic acid sequence of the primer and the primer concentration in the reaction mixture.

It should be noted that PCR amplification is a dynamic process. When using temperature to modulate the respective PCR reactions in a multiplex PCR reaction, the higher temperature annealing stage may be carried out at any temperature typically ranging from just above the lower Tm to just below the higher Tm, so long as the reaction favors production of the amplicon by the higher Tm primer set. Similarly, the annealing for the lower temperature reaction typically is at any temperature below the Tm of the low temperature primer set.

In the example provided above, in the higher temperature stage the amplicon for the low abundance RNA is amplified at a rate faster than that the amplicon for the higher abundance RNA (and preferably to the substantial exclusion of production of the second amplicon), so that, prior to the second amplification stage, where it is desirable that amplification of all amplicons proceeds in a substantially balanced manner, the amplicon for the lower abundance RNA is of sufficient abundance that the amplification of the higher abundance RNA does not interfere with the amplification of the amplicon for the lower abundance RNA. In the first stage of amplification, when the amplicon for the low abundance nucleic acid is preferentially amplified, the annealing and elongation steps may be performed above Tm₁ to gain specificity over efficiency (during the second stage of the amplification, since there is a relatively large number of low abundance target nucleic acid amplicons, selectivity no longer is a significant issue, and efficiency of amplicon production is preferred). It, therefore, should be noted that although favorable in many instances, the temperature variations may not necessarily result in the complete shutdown of one amplification reaction over another.

In another variation of the above-described amplification reaction, a first primer set with a first Tm may target a more-abundant template sequence (for instance, the control template sequence) and a second primer set with a higher Tm may target a less-abundant template sequence. In this case, the more-abundant template and the less-abundant template may both be amplified in a first stage at a temperature below the (lower) Tm of the first primer set. When a threshold amount of amplicon corresponding to the more abundant template is reached, the annealing and/or elongation temperature of the reaction is raised above the Tm of the first primer set, but below the higher Tm of the second primer set to effectively shut down amplification of the more abundant template.

Selection of three or more sets of PCR primer sets having three or more different Tms (for instance, Tm₁>Tm₂>Tm₃) can be used to amplify sequences of varying abundance in a stepwise manner, so long as the differences in the Tms are sufficiently large to permit preferential amplification of desired sequences to the substantial exclusion of undesired sequences for a desired number of cycles. In that process, the lowest abundance sequences are amplified in a first stage for a predetermined number of cycles. Next, the lowest abundance and the lesser abundance sequences are amplified in a second stage for a predetermined number of cycles. Lastly, all sequences are amplified in a third stage. As with the two-stage reaction described above, the minimum temperature for each stage may vary, depending on the relative efficiencies of each single amplification reaction of the multiplex reaction. It should be recognized that two or more amplimers may have substantially the same Tm, to permit amplification of more than one species of similar abundance at any stage of the amplification process. As with the two-stage reaction, the three-stage reaction may also proceed stepwise from amplification of the most abundant nucleic acid species at the lowest annealing temperature to amplification of the least abundant species at the highest annealing temperature.

In a further embodiment, exploitation of differences in amplicon melting temperatures between different amplicons in a given multiplexed amplification reaction permits amplification of three or more target sequences in a single reaction. By “exploitation” of the differences in amplicon Tm, it is meant selecting reaction conditions that favors balanced amplification of all multiplexed amplification reactions, including temporarily quenching production of one amplicon in favor of others. This method addresses homogeneous multiplex reactions that include 3 or more reactions in the same reaction vessel. Homogeneous, fluorescent real time PCR reactions in particular are anticipated. As a non-limiting example, in certain reactions, relative abundance of two of the target sequences, or superior efficiency of two of the reactions, can cause the a third, less abundant, or lower efficiency reaction, to be competed out completely. Furthermore, in quantitative PCR reactions it may be desirable to include a certain type of gene sequence that serves as a normalizer to which the target genes to be quantified are compared. A value is determined generally as a delta of the Ct value of the target and the Ct of the normalizer sequence. For reactions where at least one of two difference target genes must be detected to make a diagnosis, and where a normalizing sequence is included to enable relative quantification, conditions may arise to defeat the ability of the test to quantify each of the two target sequences. In a first non-limiting example, the normalizer gene is in far excess of the target gene sequence at any of the expected concentration ranges of the target, thereby reducing the sensitivity of the target gene reaction. In a second non-limiting example, a target gene may be present in much higher concentrations than the normalizer gene, such that the normalizer gene reaction is out-competed. In such a case, without properly balancing the multiplexed reaction, no Ct value is generated for the normalizer gene and the target gene cannot be quantified.

This embodiment resolves both of these example conditions by simultaneously exploiting manipulations of the binding energy of double stranded oligonucleotides to single stranded DNA or melting temperatures of the resulting amplicons. Exploitation of differences in primer Tm and/or amplicon Tm will permit three or more target sequences to be detected.

In a non-limiting example of a triplex reaction, the first and second target sequences are specific diagnostic targets, and the third is a housekeeping gene (endogenous control) for quantitative normalization. The concentration of the first gene sequence may range from zero to very high concentrations that may quench the second two reactions. The housekeeping gene always is present at moderate or high concentrations and may quench the second target sequence reaction. The goal therefore is to design an assay that prevents the first gene sequence reaction from quenching the other two reactions and also prevents the housekeeping gene from quenching the second reaction. In this case, a Ct value should always be generated for the housekeeping gene regardless of the concentrations of either target sequence, and a Ct value for the second target sequence should be generated even when the housekeeping gene is present at high concentration. Whenever either target gene sequence is present, a Ct value should be generated and a delta Ct value determined, thereby permitting quantification of the target gene. This can be achieved by manipulating and exploiting the primer Tm and/or the amplicon 1 m of the first target gene sequence and the housekeeping gene sequence. There are two cases, in which: First reaction primer set Tm=TmP1; First reaction amplicon Tm=TmA1; Second reaction primer set Tm=TmP2; Second reaction amplicon Tm=TmA2; Third reaction primer set Tm=TmP3; and Third reaction amplicon Tm=TmA3
TmP1<<TmP2=TmP3 and TmA1=TmA2<<TmA3 Case 1
TmP1=TmP2>TmP3 and TmA1>TmA2=TmA3. Case 2

In these examples, the reactions are monitored in real-time. For case 1, in the thermal cycling protocol the low temperature (Annealing temp) is set below the Tm of all the reactions, that is, below the TmP1, and the high temp melt is set above all amplicon Tms, that is, above the TmA3. If the first gene sequence is present in high abundance, a relatively early Ct value will be detected. At that point in time, or cycle number, the thermal cycling protocol low temperature is raised to be above the TmP1 but still below the TmP2 and TmP3, thereby effectively shutting off, or quenching, the first target reaction, allowing the other two reactions to proceed. If the first gene sequence is not present in high abundance, the Ct value is not detected, and the thermal cycling protocol low temp is not changed. Thermal cycling simply continues until a Ct value for the housekeeping gene is detected. At this point in the reaction or cycle number, the high temp (melting) is lowered to below TmA3, but still above the TmA1 and TmA2. Under these reaction conditions, the housekeeping gene amplicons no longer melt at each cycle, and the housekeeping gene reaction is effectively shut off. This allows the lower abundance second target gene sequence reaction to continue without completion by the housekeeping gene reaction.

For Case 2, the thermal cycling protocol low temp annealing is set below the Tm of primer set TmP3 and the high temp melt is set above all amplicons, that is, above TmA1.

In some cases, it is possible that it is not predictable which of two targets (PI, P2, A1, and A2) may be present in higher abundance vs. the control (P3 and A3). So additional cases are as follows:
TmP1=TmP2>TmP3 and TmA1=TmA2>TmA3; Case 3
or
TmP1=TmP3<TmP2 and TmA1=TmA2<TmA3 Case 4

For case 3, if either P1 and P2 are detectable before the control, (P3), then shifting to a lower amplicon melt temp will shut off the both target amplification, allowing the control gene to amplify without competition. If the control amplifies first, then the annealing temperature is shifted up, effectively shutting off the control reaction. As a non-limiting example, primers can be designed to have a Tm of 70° C. with the Tm of the control being 64° C. The amplicon Tm for the targets is 85° C. and for the control, 80° C. This scenario allows a cycle threshold value to be determined in all cases of the control gene and for the target genes if they are present, allowing the calculation of a delta Ct value and generating a relative quantification value.

In case 4, amplification of the control is controlled by manipulation of the amplicon melt temperature and the targets are controlled by primer annealing Tms.

As illustrated in a non-limiting example below, two target genes (TACSTD1 and PIP) were selected as markers for breast cancer detection in sentinel lymph nodes and one housekeeping gene (β-GUS) was selected as an endogenous control (endogenous control). An assay was developed in which these three genes were detected in a multiplex assay. As an option, a fourth reaction for amplification of a synthetic Internal Positive Control is contemplated. If this is needed, the multiplex reaction will be more complex than triplex. In multiplex PCR reactions, if one gene signal is detected more than 4 cycles before another, the efficiency of the second reaction is greatly impaired. To avoid false negative results, as described above, two “quenching” strategies have been exploited for use in this assay. Both strategies rely on differential design of primer pairs and “Advance to Next Stage (ANS)” capabilities within the software of the instrument (for example the GeneXpert® or SmartCycler instruments). In order to combine the two quenching strategies, a “Bifurcated ANS” approach would be needed as described below.

For the purposes herein, the two (bifurcated) strategies will be called “Anneal” and “Amplicon.” In the Anneal strategy, the primer pairs for two or more of the three genes in, for example and without limitation, a triplex reaction are designed to have a difference in anneal temperatures such that, for example, the endogenous control primers can anneal at 68° C. and the target gene primers can anneal at 60° C. In this non-limiting example, if either one of the target gene signals is detected before the endogenous control gene, ANS is used to change the cycling parameters to a higher anneal temperature. This prevents further amplification of both target genes but allows for high efficiency amplification of the lower abundance endogenous control gene.

In the “Amplicon” strategy, the primer pairs for three genes in, for example and without limitation, a triplex reaction, are designed such that at least two of the resulting amplicons have different melting temperatures. For example, the target genes are designed with an amplicon Tm of ˜85° C. (or at least 4° C. higher than the endogenous control). If the endogenous control gene signal is detected before either of the target genes, ANS is used to change the cycling parameters to a lower melting temperature (between the Tm of the targets and the Tm of the endogenous control rather than the standard 95° C.). This prevents further amplification of the target genes but allows for high efficiency amplification of the lower abundance endogenous control gene. It should be noted that the above-described assays also can be run with opposite assignments for the target genes and the endogenous control.

ANS Modifications were made to the software of both of Cepheid's SmartCycler® and GeneXpert® instruments to test and validate this approach. The ANS process according to one embodiment of the invention, comprises an automated system for time adjusting reaction conditions in a multiplex reaction to favor production of one or more amplicons in the reaction over other(s). The ANS process modulates the reaction conditions based on the relative accumulation of amplicons. In one embodiment the ANS process provides cycling at one set of temperatures to be ended prior to the specified repeat number when a reporter being monitored crosses a threshold. A protocol associated with the ANS process then advances to a new stage. All or some reporters (for example and without limitation, a fluorescent dye or dye combination) used in an assay (for example and without limitation, sample preparation steps and multiplex PCR protocols) may be monitored. The ANS process provides the following features, for example, taken alone or in combination:
(1) Monitoring of any cycling stage in any protocol;
(2) Monitoring multiple reporters in one stage. In one embodiment, threshold crossings of different reporters may advance to either the same or different stages;
(3) Performing an additional number of cycles after threshold crossing before advancing to a new stage, which can be set for each reporter;
(4) Terminating the protocol at the end of the monitoring stage if one or more, or all of the monitored reporters cross the threshold and/or fail to cross the threshold; and
(5) Terminating the protocol at the end of the new stage.

If the ANS process (for example, a software program) decides to advance to a new stage, it will first complete the current cycle. If the advance to new stage condition is detected after the system is already in a different stage, the software ignores the advances to the new stage event.

In one embodiment, the ANS process records the information related to the advance to new stage event and generates the following reports, for example:
(1) The module name affected;
(2) The reporter name and the Ct value;
(3) The required protocol number, the monitoring stage number, the updated repeat count and the new stage number; and
(4) The number of repeat count added or skipped for this request.

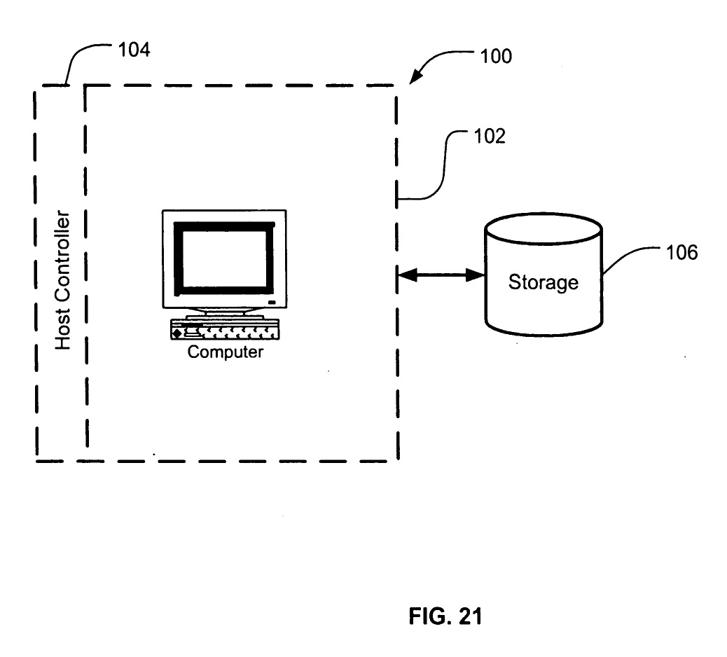

FIG. 21 illustrates one embodiment of a system 100 for implementing the ANS process described above. The system 100 may include a device 102 operating under the command of a controller 104. The broken lines are intended to indicate that in some implementations, the controller 104, or portions thereof considered collectively, may instruct one or more elements of the device 102 to operate as described. Accordingly, the functions associated with the ANS process described herein may be implemented as software executing in the system 100 and controlling one or more elements thereof. An example of a device 102 in accordance with one embodiment of the present invention is a general-purpose computer capable of responding to and executing instructions in a defined manner. Other examples include a special-purpose computer including, for example, a personal computer (PC), a workstation, a server, a laptop computer, a web-enabled telephone, a web-enabled personal digital assistant (PDA), a microprocessor, an integrated circuit, an application-specific integrated circuit, a microprocessor, a microcontroller, a network server, a Java virtual machine, a logic array, a programmable logic array, a micro-computer, a mini-computer, or a large frame computer, or any other component, machine, tool, equipment, or some combination thereof capable of responding to and executing instructions. In one embodiment, system 100 may be implemented in a programmable thermal cycling device, or a device containing a thermal cycler, such as, without limitation, Cepheid's SmartCycler® and GeneXpert® systems as well as Applied Biosystems PRISM line of thermal cycling equipment. Furthermore, the system 100 may include a central processing engine including a baseline processor, memory, and communications capabilities. The system 100 also may include a communications system bus to enable multiple processors to communicate with each other. In addition, the system 100 may include a storage 106 in the form of a disk drive, cartridge drive, and control elements for loading new software. In embodiments of the invention, one or more reference values may be stored in a memory associated with the device 102.

Embodiments of the controller 104 may include, for example, a program, code, a set of instructions, or some combination thereof, executable by the device 102 for independently or collectively instructing the device 102 to interact and operate as programmed. One example of a controller 104 is a software application (for example, operating system, browser application, client application, server application, proxy application, on-line service provider application, and/or private network application) installed on the device 102 for directing execution of instructions. In one embodiment, the controller 104 may be a Windows™ based operating system. The controller 104 may be implemented by utilizing any suitable computer language (e.g., C\C++, UNIX SHELL SCRIPT, PERL, JAVA, JAVASCRIPT, HTML/DHTML/XML, FLASH, WINDOWS NT, UNIX/LINUX, APACHE, RDBMS including ORACLE, INFORMIX, and MySQL) and/or object-oriented techniques.

In one embodiment, the controller 104 may be embodied permanently or temporarily in any type of machine, component, physical or virtual equipment, storage medium, or propagated signal capable of delivering instructions to the device 102. In particular, the controller 104 (e.g., software application, and/or computer program) may be stored on any suitable computer readable media (e.g., disk, device, or propagated signal), readable by the device 102, such that if the device 102 reads the storage medium, the functions described herein are performed. For example, in one embodiment, the controller 104 may be embodied in various computer-readable media for performing the functions associated with the ANS process.

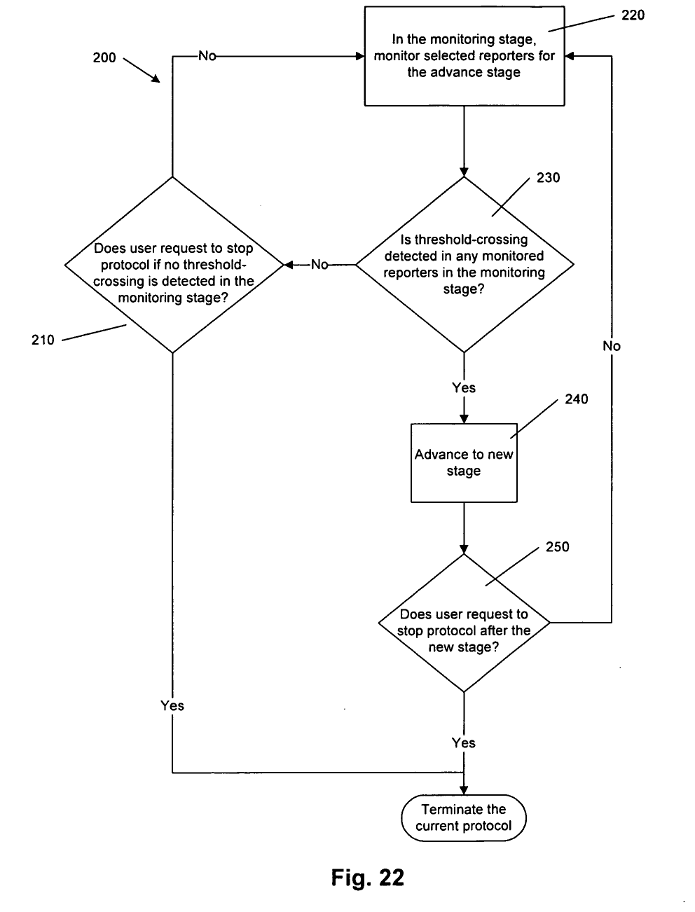

FIG. 22 illustrates one embodiment of a flow diagram 200 for implementing the functions associated with the ANS process described above. At decision step 210, the process determines whether the user requests to stop protocol if no threshold crossing is detected in the monitoring stage. If no threshold crossing is detected, at step 220, in the monitoring stage, the process monitors selected reporters for the advance stage. The process continues at decision step 230, where it decides whether threshold crossing is detected in any monitored reporters in the monitoring stage. If no threshold crossing is detected in any monitored reporters in the monitoring stage, the process continues to step 210 and, if the user does not request to stop the protocol if no threshold crossing is detected in the monitoring stage, the process repeats 210-230 in an iterative manner until at step 230, a threshold crossing is detected in anyone of the monitored reporters in the monitoring stage. The process then continues to step 240 where the process advances to a new stage. At decision step 250, the process determines whether the user requests to stop the protocol after the new stage. If the user does not request to stop the protocol after the new stage, the process continues to step 220. Steps 210-250 are repeated iteratively until the current protocol is terminated at step 260 if at decision step 210, the user requests to stop protocol if no threshold crossing is detected in the monitoring stage or if at decision step 250, the user requests to stop the protocol after the new stage.

Threshold crossing may be determined by monitoring a reporter (for example, fluorescent probe) to determine if the signal from the reporter exceeds a user-defined or default fluorescent value. Alternatively, a noise-based threshold may be determined (for example, two or three times the standard deviation of the noise) and the reporter monitored for crossing of the noise-based threshold. In other embodiments, threshold crossing may be determined using a derivative of the amplification growth curve. These and other techniques for determining threshold crossing are described in U.S. Pat. No. 6,783,934 the disclosure of which is incorporated by reference herein.

In one embodiment of the multiplexed amplification methods described herein, sentinel lymph nodes are excised at the time of tumor excision. The lymph nodes are frozen in OCT solution and thin sections are cut. Twenty to twenty-five sections typically are used for analysis. The amount of tissue present in a given sample is highly variable due, without limitation, to variation in lymph node size and sectioning techniques. The calculations needed for determining whether a given sample is positive or negative for cancer cells is therefore based on target gene expression levels relative to endogenous control gene expression. In one embodiment, the assay design uses β-GUS for the endogenous control gene. PIP and TACSTD1 are the two target genes for the assay.

When one or both target mRNA levels are higher than endogenous control, endogenous control amplification does not occur. Therefore, it is important to “quench” the target amplification to allow endogenous control to amplify. When both target mRNA levels are lower than endogenous control, target gene amplification does not occur. In that case it is important to “quench” endogenous control amplification to allow target amplification.

In preliminary studies, in the amplicon limitation method, without ANS software running, β-GUS was not quenched at all for 60 cycles and PIP was quenched at 40 cycles. In the amplicon-limitation method with ANS, β-GUS was not quenched over 60 cycles and PIP was quenched 1 cycle after the threshold was reached. In the primer temperature-dependence assay (anneal), PIP was not quenched at all over 60 cycles, while β-GUS was quenched three cycles after the threshold was reached.

In cancer-positive samples, depending upon the number of cancer cells present in the lymph node sample, the level of expression of the two target genes is highly variable, ranging from higher than the endogenous control several fold lower than the endogenous control. For example, TACSTD1 is expressed at very low levels in normal lymph node tissue. Therefore, there is a need for accurate ΔCt values. Further complicating the assay is the frequency of high target expression. For example, in 10-15% of the samples, PIP levels are higher than β-GUS levels and in approximately 30% of the samples, TACSTD1 levels are higher than β-GUS levels. For that reason, ΔCt values can range from significantly positive values to significantly negative values for positive samples. Thus, if endogenous control is not detected, there is no rational way to set a cutoff for calling a sample positive or negative due to the inherent variability of sample size, resulting in indeterminate results. If target expression is low even though the sample is a true positive, unless the endogenous control is quenched, the sample would be a false negative.

Taking the example of TACSTD1 and PIP as target genes, there are three options to overcome the described problems. One option would be to replace β-GUS with a different endogenous control gene, such as O₂ microglobulin (β2M, having higher expression levels than β-GUS), though there still remains the possibility of encountering a sample where the target genes are amplified before the endogenous control. Other potential endogenous controls include P-Actin (ACTB), gluteraldehyde-3-phosphate dehydrogenase (GAPD) and peptidyl-prolyl isomerase A (PPIA). Nevertheless, there are certain limitations to the choice of other endogenous controls. As mentioned above, one good candidate gene to replace β-GUS is β2M. Some studies demonstrate inherent variability in O₂M expression in normal lymph nodes making ΔCt determinations unpredictable. Alternate housekeeping genes with higher expression than β-GUS, such as ACTB, GAPD and PPIA, result in unequal RT efficiencies—thus, the ΔCt values likely would be unreliable.

In a second option, for example and without limitation, the target primers anneal at Tm=68° C. and the target amplicons denature at Tm=86° C., while the endogenous control primers anneal at Tm=58-60° C. and the endogenous control amplicon denatures at Tm=80° C. (target primer Tm>control primer Tm and target amplicon Tm>control amplicon Tm), permitting exploitation of the differences in primer and amplicon Tm to balance the reaction.

In a third option, for example and without limitation, the target primers anneal at Tm=58-60° C. and the target amplicons denature at Tm=80° C., while the endogenous control primers anneal at Tm=68° C. and the endogenous control amplicon denatures at Tm=86° C. (target primer Tm<control primer Tm and target amplicon Tm<control amplicon Tm), also permitting exploitation of the differences in primer and amplicon Tm to balance the reaction.

The primer annealing temperatures and amplicon denaturation temperatures are provided above for example only, and are not intended to limit in any way the potential choices of Tms for the various primers an amplicons in a multiplexed nucleic aid amplification reaction, so long as the differences in Tms can be exploited in a nucleic acid amplification reaction, they are contemplated to be useful in the present invention.

By the amplification method described above, an additional tool is provided for the balancing of multiplex PCR reactions besides the standard matching of Tms and using limiting amounts of one or more PCR primer sets. The exploitation of PCR primer sets and amplicons with different Tms as a method for sequentially amplifying different amplicons may be preferred in certain circumstances to the sequential addition of additional primer sets. However, the use of temperature-dependent sequencing of multiplex PCR reactions may be coupled with the sequential physical addition of primer sets to a single reaction mixture.

An internal positive control that confirms the operation of a particular amplification reaction for a negative result also may be used. Internal positive controls (IPCs) are DNA oligonucleotides that have the same primer sequences as the target gene (for example, PIP or TACSTD1) but have a different internal probe sequence. Selected sites in an IPC optionally may be synthesized with uracil instead of thymine so that contamination with the highly concentrated mimic could be controlled using uracil DNA glycosylase, if required. The IPCs maybe added to any PCR reaction master mix in amounts that are determined empirically to give Ct values typically greater than the Ct values of the endogenous target of the primer set. The PCR assays are then performed according to standard protocols, and even when there is no endogenous target for the primer set, the IPC would be amplified, thereby verifying that the failure to amplify the target endogenous DNA is not a failure of the PCR reagents in the master mix. In one embodiment, an IPC probe fluoresces differently than the probe for the endogenous sequences. A variation of this for use in RT-PCR reactions is where the IPC is an RNA and the RNA includes an RT primer sequence. In this embodiment, the IPC verifies function of both the RT and PCR reactions. Both RNA and DNA IPCs (with different corresponding probes) may also be employed to differentiate difficulties in the RT and PCR reactions.

The rapid QRT-PCR protocols described herein may be run in about 20 minutes. This short time period permits the assay to be run intraoperatively so that a surgeon can decide on a surgical course during a single operation (typically the patient will remain anesthetized and/or otherwise sedated in a single “operation,” though there may be a waiting period between when the sample to be tested is obtained and the time the intraoperative assay is complete), rather than requiring a second operation, or requiring the surgeon to perform unneeded or overly broad prophylactic procedures. For instance, in the surgical evaluation of certain cancers, including breast cancer, melanoma, lung cancer, esophageal cancer and colon cancer, tumors and sentinel lymph nodes are removed in a first operation. The sentinel nodes are later evaluated for micrometastases, and, when micrometastases are detected in a patient's sentinel lymph node, the patient will need a second operation, thereby increasing the patient's surgical risks and patient discomfort associated with multiple operations. With the ability to determine the expression levels of certain tumor-specific markers described herein in less than 30 minutes with increased accuracy, a physician can make an immediate decision on how to proceed without requiring the patient to leave the operating room or associated facilities. The rapid test also is applicable to needle biopsies taken in a physician's office. A patient need not wait for days to get the results of a biopsy (such as a needle biopsy of a tumor or lymph node), but can now get more accurate results in a very short time.

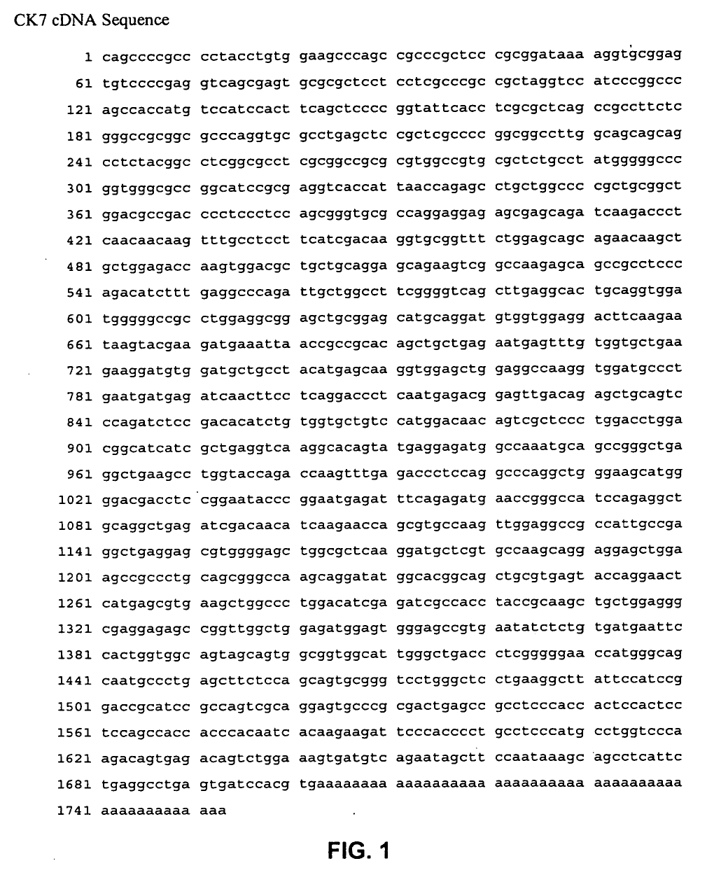

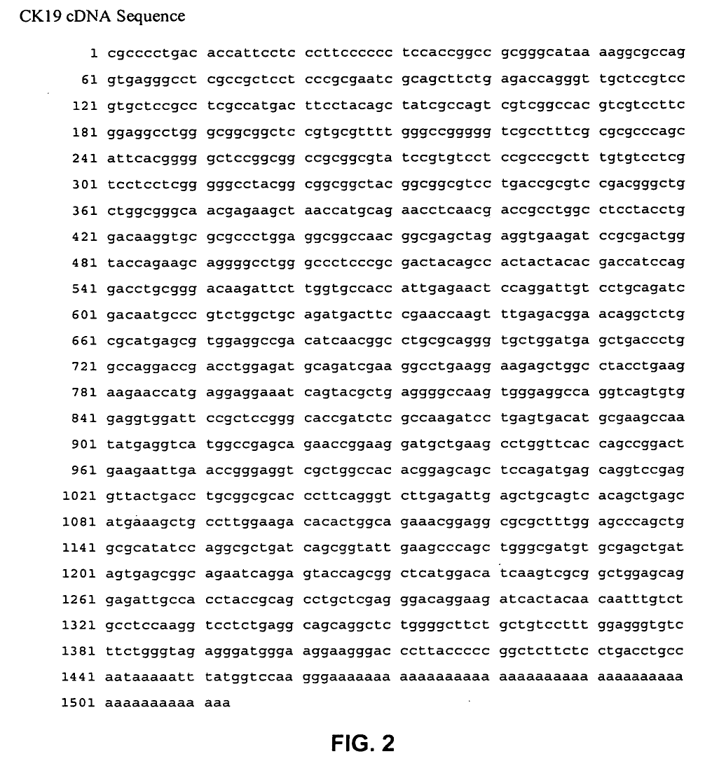

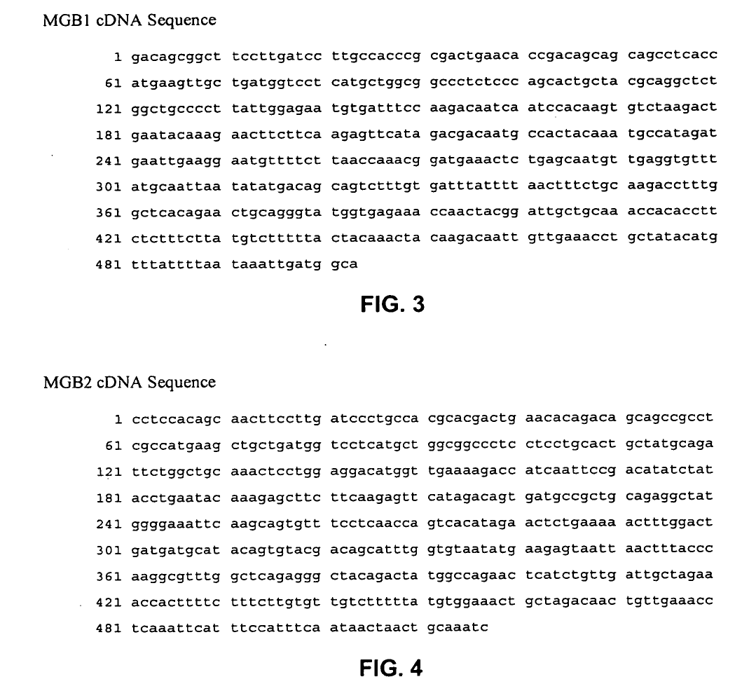

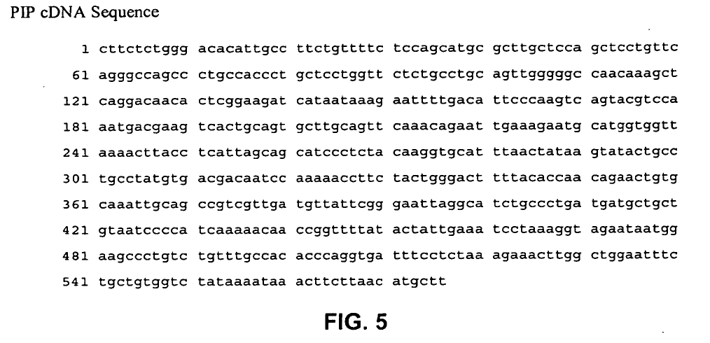

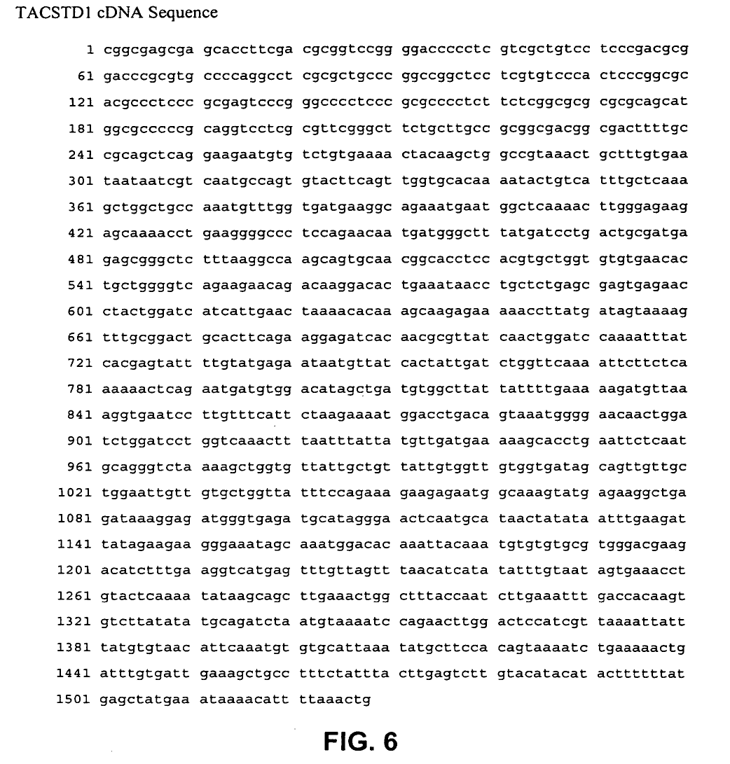

Table B provides primer and probe sequences for the mRNA quantification assays described and depicted in the Examples and Figures. FIGS. 1-6 provide non-limiting examples of cDNA sequences of the various mRNA species detected in the Examples. Although the sequences provided in Table B were found effective in the assays described in the examples, other primers and probes would likely be equally suited for use in the QRT-PCR and other mRNA detection and quantification assays, either described herein or as are known in the art. Design of alternate primer and probe sets for PCR assays, as well as for other mRNA detection assays is well within the abilities of one of average skill in the art. For example and without limitation, a number of computer software programs will generate primers and primer sets for PCR assays from cDNA sequences according to specified parameters. Non limiting examples of such software include, NetPrimer and Primer Premier 5, commercially available from PREMIER Biosoft International of Palo Alto, Calif., which also provides primer and probe design software for molecular beacon and array assays. Primers and/or probes for two or more different mRNAs can be identified, for example and without limitation, by aligning the two or more target sequences according to standard methods, determining common sequences between the two or more mRNAs and entering the common sequences into a suitable primer design computer program.

In the commercialization of the methods described herein, certain kits for detection of specific nucleic acids will be particularly useful. A test kit typically comprises one or more reagents, such as, without limitation, nucleic acid primers or probes, packaged in a container, such as, without limitation, a vial, tube or bottle, in a package suitable for commercial distribution, such as, without limitation, a box, a sealed pouch, a blister pack and a carton. The package typically contains a label or packaging insert indicating that the packaged reagents can be used in a method for identifying expression or markers indicative of the presence of cancer cells in a lymph node of a patient. As used herein, “packaging materials” includes any article used in the packaging for distribution of reagents in a kit, including without limitation containers, vials, tubes, bottles, pouches, blister packaging, labels, tags, instruction sheets and package inserts. One example of such a kit would include reagents necessary for the one-tube QRT-PCR process described above. In one example, the kit would include the above-described reagents, including reverse transcriptase, a reverse transcriptase primer, a corresponding PCR primer set, a thermostable DNA polymerase, such as Taq polymerase, and a suitable fluorescent reporter, such as, without limitation, a probe for a fluorescent 5′ nuclease assay, a molecular beacon probe, a single dye primer or a fluorescent dye specific to double-stranded DNA, such as ethidium bromide. The primers may be present in quantities that would yield the high concentrations described above. Thermostable DNA polymerases are commonly and commercially available from a variety of manufacturers. Additional materials in the kit may include: suitable reaction tubes or vials, a barrier composition, typically a wax bead, optionally including magnesium; reaction mixtures (often concentrated, for example 2×, 5×, 10× or 20×) for the reverse transcriptase and the PCR stages, including necessary buffers and reagents such as dNTPs; nuclease- or RNase-free water; RNase inhibitor; control nucleic acid(s) and/or any additional buffers, compounds, co-factors, ionic constituents, proteins and enzymes, polymers, and the like that may be used in reverse transcriptase and/or PCR stages of QRT-PCR reactions.

Components of a kit are packaged in any manner that is commercially practicable. For example, PCR primers and reverse transcriptase may be packaged individually to facilitate flexibility in configuring the assay, or together to increase ease of use and to reduce contamination. Similarly, buffers, salts and co-factors can be packaged separately or together.

The kits also may include reagents and mechanical components suitable for the manual or automated extraction of nucleic acid from a tissue sample. These reagents are known to those skilled in the art and typically are a matter of design choice. For instance, in one embodiment of an automated process, tissue is disrupted ultrasonically in a suitable lysis solution provided in the kit. The resultant lysate solution is then filtered and RNA is bound to RNA-binding magnetic beads also provided in the kit or cartridge. The bead-bound RNA is washed, and the RNA is eluted from the beads and placed into a suitable reverse transcriptase reaction mixture prior to the reverse transcriptase reaction. In automated processes, the choice of reagents and their mode of packaging (for instance in disposable single-use cartridges) typically are dictated by the physical configuration of the robotics and fluidics of the specific RNA extraction system, for example and without limitation, the GeneXpert® system. International Patent Publication Nos. WO 04/48931, WO 03/77055, WO 03/72253, WO 03/55973, WO 02/52030, WO 02/18902, WO 01/84463, WO 01157253, WO 01/45845, WO 00/73413, WO 00/73412 and WO 00/72970 provide non-limiting examples of cartridge-based systems and related technology useful in the methods described herein.

The constituents of the kits may be packaged together or separately, and each constituent may be presented in one or more tubes or vials, or in cartridge form, as is appropriate. The constituents, independently or together, may be packaged in any useful state, including without limitation, in a dehydrated, lyophilized, glassified or aqueous state. The kits may take the physical form of a cartridge for use in automated processes, having two or more compartments including the above-described reagents. Suitable cartridges are disclosed for example in U.S. Pat. Nos. 6,440,725, 6,431,476, 6,403,037 and 6,374,684. As mentioned above, PCR-based technologies may be used to quantify mRNA levels in a given tissue sample. Other sequence-specific nucleic acid quantification methods may be more or less suited, so long as differences in Tm between reagents can be effectively exploited to balance the multiplexed reaction. As is understood in the field of the present invention, many variations to the standard multiplexed “PCR” reaction are known, and many exist in which differences in amplicon denaturation temperatures and/or differences in primer anneal temperatures can be exploited with the goal of balancing the multiplex reaction. Those nucleic acid amplification methods are considered to be within the scope of the present invention.

### EXAMPLE 1

### General Materials and Methods

Identification of Potential Markers. An extensive literature and public database survey was conducted to identify any potential markers. Resources for this survey included PubMed, OMIM, UniGene (http://www.ncbi.nlm.nih.gov/), GeneCards (http://bioinfo.weizmann.ac.il/cards), and CGAP (http://cgap.nci.nih.gov). Survey criteria were somewhat flexible but the goal was to identify genes with moderate to high expression in tumors and low expression in normal lymph nodes. In addition, genes reported to be up-regulated in tumors and genes with restricted tissue distribution were considered potentially useful. Finally, genes reported to be cancer-specific, such as the cancer testis antigens and hTERT, were evaluated.

Tissues and Pathological Evaluation. Tissue specimens were obtained from tissue banks at the University of Pittsburgh Medical Center through IRB approved protocols. All specimens were snap frozen in liquid nitrogen and later embedded in OCT for frozen sectioning. Twenty 5-micron sections were cut from each tissue for RNA isolation. In addition, sections were cut and placed on slides for H&E and IHC analysis at the beginning, middle (between the tenth and eleventh sections for RNA), and end of the sections for RNA isolation. All three H&E slides from each specimen underwent pathological review to confirm presence of tumor, percentage of tumor, and to identify the presence of any contaminating tissues. All of the unstained slides were stored at −20° C. Immunohistochemistry evaluation was performed using the AE1/AE3 antibody cocktail (DAKO, Carpinteria, Calif.), and Vector Elite ABC kit and Vector Aendogenous control Chromagen (Vecta Laboratories, Burlingame, Calif.). IHC was used as needed as needed to confirm the H&E histology.

Screening Approach. The screening was conducted in two phases. All potential markers entered the primary screening phase and expression was analyzed in 6 primary tumors and 10 benign lymph nodes obtained from patients without cancer (5 RNA pools with 2 lymph node RNA's per pool). Markers that showed good characteristics for lymph node metastasis detection passed into the secondary screening phase. The secondary screen consisted of expression analysis on 20-25 primary tumors, 20-25 histologically positive lymph nodes and 21 benign lymph nodes without cancer.

RNA Isolation and cDNA Synthesis. RNA was isolated using the RNeasy minikit (Qiagen, Valencia, Calif.) essentially as described by the manufacturer. The only modification was that the volume of lysis reagent was doubled and loaded the column in two steps. This was found to provide better RNA yield and purity, probably as a result of diluting out the OCT in the tissue sections. Reverse transcription was performed in 100 μL reaction volumes either with random hexamer priming or sequence-specific priming using a probe indicated in Table C, and Superscript II (Invitrogen, Carlsbad, Calif.) reverse transcriptase. For the primary screen, three reverse transcription reactions were performed, each with 500 ng of RNA. The cDNAs were combined and QPCR was performed using the equivalent of 20 ng RNA per reaction. For the secondary screen, the RNA input for primary tumors and positive nodes was also 500 ng. For benign nodes however, the RNA input was 2000 ng resulting in the equivalent of 80 ng RNA per QPCR reaction.

Quantitative PCR. All quantitative PCR was performed on the ABI Prism 7700 Sequence Detection Instrument (Applied Biosystems, Foster City, Calif.). Relative expression of the marker genes was calculated using the delta-CT methods previously described and with β-glucuronidase as the endogenous control gene. All assays were designed for use with 5′ nuclease hybridization probes although the primary screening was performed using SYBER Green quantification in order to save cost. Assays were designed using the ABI Primer Express Version 2.0 software and where possible, amplicons spanned exon junctions in order to provide cDNA specificity. All primer pairs were tested for amplification specificity (generation of a single band on gels) at 60, 62 and 64° C. annealing temperature. In addition, PCR efficiency was estimated using SYBER green quantification prior to use in the primary screen. Further optimization and more precise estimates of efficiency were performed with 5′nuclease probes for all assays used in the secondary screen.

A mixture of the Universal Human Reference RNA (Stratagene, La Jolla, Calif.) and RNAs from human placenta, thyroid, heart, colon, PCI13 cell line and SKBR3 cell line served as a universal positive expression control for all the genes in the marker screening process.

Quantification with SYBER Green (primary Screen). For SYBR Green I-based QPCR, each 50 μL reaction contained 1× TaqMan buffer A (Applied Biosystems), 300 nM each dNTP, 3.5 mMMgCl₂, 0.06 units μL Amplitaq Gold (Applied Biosystems), 0.25×SYBR Green I (Molecular Probes, Eugene, Oreg.) and 200 nM each primer. The amplification program comprised 2-stages with an initial 95° C. Taq activation stage for 12 min followed by 40 cycles of 95° C. denaturation for 15 s, 60, 62 or 64° C. anneal/extend for 60 s and a 10 second data collection step at a temperature 2-4° C. below the Tm of the specific PCR product being amplified (TB Morrison, et al. 1998). After amplification, a melting curve analysis was performed by collecting fluorescence data while increasing the temperature from 60° C.-95° C. over 20 minutes.

Quantification with 5′ Nuclease Probes (Secondary Screen). Probe-based QPCR was performed as described previously (Godfrey, et al., Clin. Cancer Res. 2001 December, 7(12):4041-8). Briefly, reactions were performed with a probe concentration of 200 nM and a 60 second anneal/extend phase at 60° C., 62° C., or 64° C. The sequences of primers and probes (purchased from IDT, Coralville, Iowa) for genes evaluated in the secondary screen are listed in Table B, below.

Data Analysis. In the primary screen, data from the melt curve was analyzed using the ABI Prism 7700 Dissociation Curve Analysis 1.0 software (Applied Biosystems). The first derivative of the melting cure was used to determine the product Tm as well as to establish the presence of the specific product in each sample. In general, samples were analyzed in duplicate PCR reactions and the average Ct value was used in the expression analysis. However, in the secondary screen triplicate reactions were performed for each individual benign node and the lowest Ct value was used in the calculation of relative expression in order to obtain the highest value of background expression for the sample.

Cancer tissue-specific studies have been conducted, as described in the Examples below, in which a variety of molecular markers were identified as correlating with pathological states in cancers including breast cancer and lung cancer. Table A identifies genes used in the following studies. Table B provides PCR primer and TAQMAN probe sequences used in the quantitative PCR and RT-PCR amplifications described herein. Table C provides RT primer sequences as used instead of random hexamer primers. All PCR and RT-PCR reactions were conducted using standard methods. For all figures, T=primary tumor; PN=tumor-positive lymph nodes (by histological screening, that is, by review of H&E stained tissue and, when needed, by IHC, as described above); and BN=benign lymph nodes (by histological screening)

TABLE A

| | | Official | | Alternative | |
|---|---|---|---|---|---|
| | Accession No./ | Gene | Official Gene | Gene | |
| Marker | OMIMNo.* | Symbol | Name | Symbol | Alias |
| CK7 | NM_005556/148059 | KRT7 | keratin 7 | K7, CK7, | Sarcolectin; |
| | | | | SCL, K2C7, | cytokeratin 7; |
| | | | | MGC3625 | type II mesothelial |
| | | | | | keratin K7; |
| | | | | | keratin, type II |
| | | | | | cytoskeletal 7; |
| | | | | | keratin, 55 K type II |
| | | | | | cytoskeletal; |
| | | | | | keratin, simple |
| | | | | | epithelial type I, K7 |
| CK19 | NM_002276/148020 | KRT19 | keratin 19 | K19, CK19, | cytokeratin 19; |
| | | | | KICS, | keratin, type I, 40-kd; |
| | | | | MGC15366 | keratin, type I |
| | | | | | cytoskeletal 19; |
| | | | | | 40-kDa keratin |
| | | | | | intermediate filament |
| | | | | | precursor gene |
| MGB1 | NM_002411/605562 | SCGB2A2 | secretoglobin, | MGB1, UGB2 | mammaglobin 1 |
| | | | family 2A. | | |
| | | | member 2 | | |
| MGB2 | NM_002407/604398 | SCGB2A1 | secretoglobin, | LPHC, MGB2, | lipophilin C; |
| | | | family 2A. | UGB3 | mammaglobin 2; |
| | | | member 1 | | mammaglobin B |
| PIP | NM_002652/176720 | PIP | prolactin- | GPI7, | prolactin-inducible |
| | | | induced protein | GCDFP-15 | protein |
| TACSTD1 | NM_00354/185535 | TACSTD1 | tumor-associated | EGP; KSA; | MK-1 antigen; |
| | | | calcium signal | M4S1; MK-1; | antigen identified by |
| | | | transducer 1 | KS1/4; | monoclonal |
| | | | | EGP40; | antibody AUA1; |
| | | | | MIC18; | membrane component, |
| | | | | TROP1; Ep- | chromosome 4, surface |
| | | | | CAM; CO17- | marker (35 kD |
| | | | | 1A; GA733-2 | glycoprotein) |

*Online Mendelian Inheritance in Man (www.ncbi.nlm.nih.gov).

TABLE B

Oligonucleotide primer and probe
sequences used in secondary marker
screening for all cancer types

| | | | |
|---|---|---|---|
| | Oligonu- | | Sequence |
| Gene | cleotide | Sequence (5′-3′) | Reference |

| | | | |
|---|---|---|---|
| CK19 | Forward | AGATCGACAACGCCCGT, | SEQ ID NO: 2 |
| | primer | | bases 596 to 612 |
| | Reverse | AGAGCCTGTTCCGTCTCAAA | SEQ ID NO: 7 |
| | primer | | |
| | Probe | TGGCTGCAGATGACTTCCGA | SEQ ID NO: 2 |
| | | ACCA, | bases 614 to 637 |
| CK7 | Forward | CCCTCAATGAGACGGAGTTG | SEQ ID NO: 1 |
| | primer | A, | bases 807 to 827 |
| | Reverse | CCAGGGAGCGACTGTTGTC | SEQ ID NO: 8 |
| | primer | | |
| | Probe | AGCTGCAGTCCCAGATCTC | SEQ ID NO: 1 |
| | | CGACACATC, | bases 831 to 858 |
| MGB1 | Forward | GTTGCTGATGGTCCTCAT | SEQ ID NO: 3 |
| | | GCT, | bases 66 to 86 |
| | Reverse | GGAAATCACATTCTCCAATAA | SEQ ID NO: 9 |
| | primer | GGG | |
| | Probe | AGCCAGAGCCTGCGTAGCAGT | SEQ ID NO: 10 |
| | | GCT | |
| MGB2 | Forward | ATGCCGCTGCAGAGGCTAT, | SEQ ID NO: 4 |
| | primer | | bases 222 to 240 |
| | Reverse | CTGTCGTACACTGTATGCA | SEQ ID NO: 11 |
| | primer | TCATCA | |
| | Probe | TCAAGCAGTGTTTCCTCAACC | SEQ ID NO: 4 |
| | | AGTCACA, | bases 249 to 276 |
| PIP | Forward | CTGGGACTTTTACACCAACAG | SEQ ID NO: 5 |
| | primer | AACT, | bases 333 to 357 |
| | Reverse | GCAGATGCCTAATTCCCGAA | SEQ ID NO: 12 |
| | primer | | |
| | Probe | TGCAAATTGCAGCCGTCGTTG | SEQ ID NO: 5 |
| | | ATGT, | bases 386 to 405 |
| TAC- | Forward | TCATTTGCTCAAAGCTGG | SEQ ID NO: 6 |
| STD1 | primer | CTG, | bases 348 to 368 |
| | Reverse | GGTTTTGCTCTTCTCCCAAG | SEQ ID NO: 13 |
| | primer | TTT | |
| | Probe | AAATGTTTGGTGATGAAGGCA | SEQ ID NO: 6 |
| | | GAAATGAATGG, | bases 371 to 402 |

TABLE C

| Gene | | Sequence |
|---|---|---|
| Marker | RT Specific Primer (5′→3′) | Reference |
| MGB1 | GGAAATCACATTCTCCAAT | SEQ ID NO: 14 |
| PIP | GCAGATGCCTAATTCCC | SEQ ID NO: 15 |
| TACSTD1 | AGCCCATCATTGTTCTG | SEQ ID NO: 16 |

### Example 2

### Breast Cancer

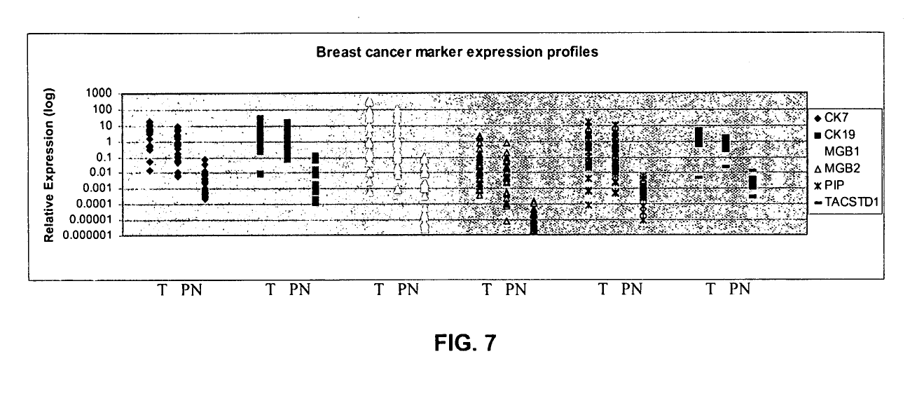

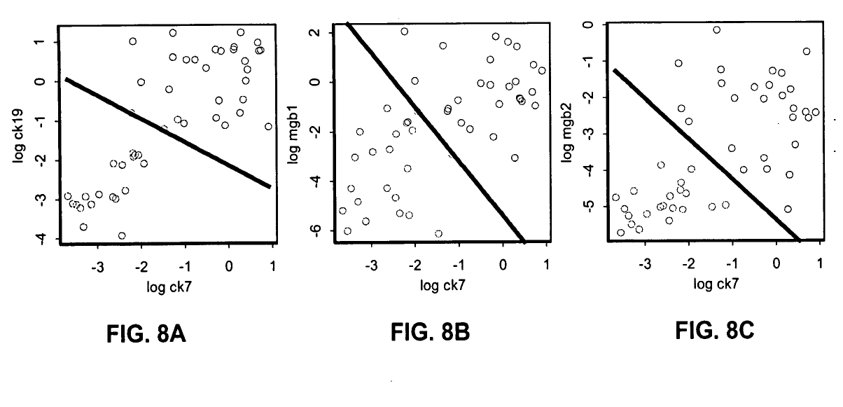

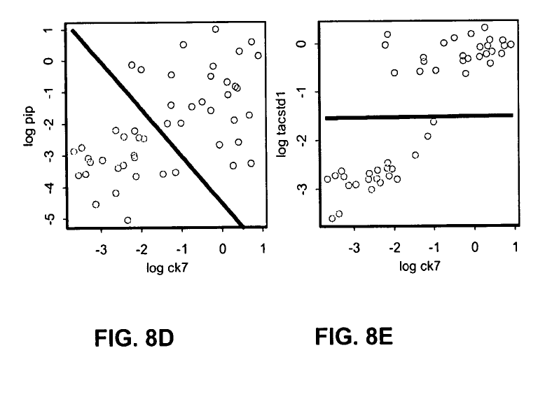

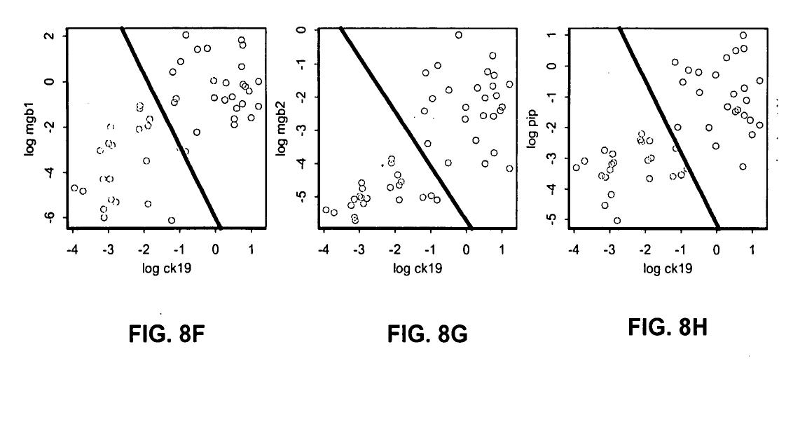

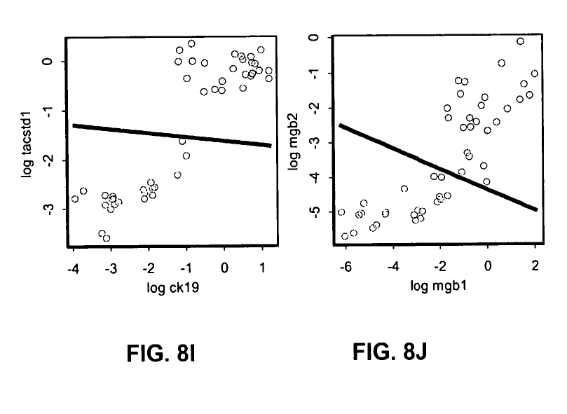

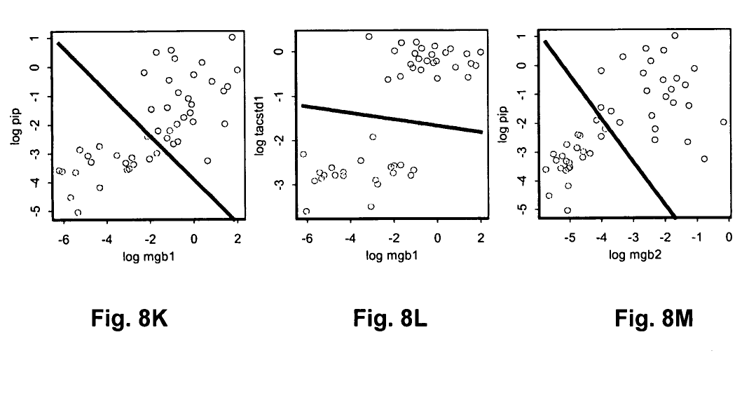

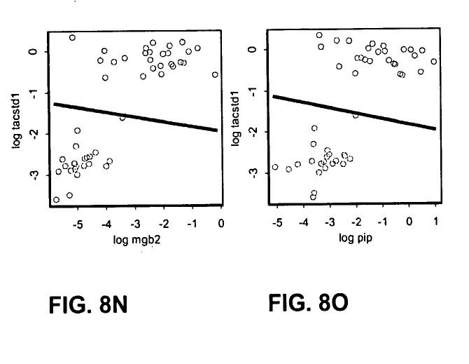

Expression levels of CK7, CK19, MGB1, MGB2, PIP, and TACSTD1 were determined by the methods described in Example 1. FIG. 7 is a scatter plot showing the expression levels of CK7, CK19, MGB1, MGB2, PIP, and TACSTD1 in primary tumor, tumor-positive lymph nodes and benign lymph nodes. FIGS. 8A-O provide scatter plots illustrating the ability of two-marker systems to distinguish between benign and malignant cells in a lymph node. Tables D and E provide the raw data from which the graphs of FIGS. 7 and 8A-O were generated. This data illustrates the strong correlation of expression of CK7, CK19, MGB1, MGB2, PIP, and TACSTD1 markers, alone or in combination, in sentinel lymph nodes with the presence of malignant cells arising from a breast cancer in the sentinel lymph nodes.

TABLE D
Single Marker Prediction Characteristics for Breast Cancer

| | Observed Data | | | Parametric Bootstrap Estimates* | | | |
|---|---|---|---|---|---|---|---|
| | | | Classification | | | Classification | Classification |
| Marker | Sensitivity | Specificity | Accuracy | Sensitivity | Specificity | Accuracy | Bias** |
| CK7 | .889 | .952 | .917 | .828 | .909 | .863 | .054 |
| CK19 | 1.0 | .952 | .979 | .997 | .891 | .951 | .028 |
| MGB1 | .926 | .857 | .896 | .903 | .748 | .836 | .060 |
| MGB2 | .963 | .905 | .938 | .943 | .834 | .895 | .043 |
| PIP | .852 | .952 | .896 | .814 | .892 | .848 | .048 |
| TACSTD1 | 1.0 | 1.0 | 1.0 | .999 | .956 | .980 | .020 |
| 500 parametric bootstrap samples of 48 lymph node expression levels (27 positive, 21 benign were generated from the log-normal distribution and a new decision rule based on the most accurate cutoff was formulated each time (total of 500 bootstrap decision rules). The differences between classifying the original data and classifying the bootstrap data were averaged to form the estimate of bias in the re-substitution decision rule. The respective | | | | | | | |
| # estimated bias was then subtracted from the sensitivity, specificity and classification of the original data to arrive at the bootstrap estimates. The bias in the estimated classification accuracy is shown in the last column | | | | | | | |

**Classification Bias = average difference in classification accuracies of the bootstrap decision rule applied to original data and the bootstrap decision rule applied to the bootstrap data. This estimates the optimism in using the original data to characterize the decision rule.

TABLE E
Two Marker Prediction Characteristics for Breast Cancer

| | Observed Data | | | Parametric Bootstrap Estimates* | | | |
|---|---|---|---|---|---|---|---|
| | | | Classification | | | Classification | Classification |
| | Sensitivity | Specificity | Accuracy | Sensitivity | Specificity | Accuracy | Bias** |
| CK7 + CK19 | 1.0 | .905 | .958 | .993 | .868 | .938 | .020 |
| CK7 + MGB1 | .963 | 1.0 | .979 | .954 | 1.0 | .977 | .002 |
| CK7 + MGB2 | 1.0 | 1.0 | 1.0 | 1.0 | 1.0 | 1.0 | .000 |
| CK7 + PIP | .963 | 1.0 | .979 | .963 | 1.0 | .979 | .000 |
| CK7 + TACSTD1 | .963 | 1.0 | .979 | .928 | 1.0 | .959 | .020 |
| CK19 + MGB1 | 1.0 | 1.0 | 1.0 | .996 | 1.0 | 1.0 | .000 |
| CK19 + MGB2 | .963 | 1.0 | .979 | .945 | 1.0 | .975 | .004 |
| CK19 + PIP | .926 | 1.0 | .958 | .900 | 1.0 | .951 | .007 |
| CK19 + TACSTD1 | .963 | 1.0 | .979 | .928 | 1.0 | .960 | .019 |
| MGB1 + MGB2 | .889 | .952 | .917 | .853 | .925 | .885 | .032 |
| MGB1 + PIP | .963 | .905 | .938 | .963 | .876 | .934 | .004 |
| MGB1 + TACSTD1 | .963 | 1.0 | .979 | .942 | 1.0 | .967 | .012 |
| MGB2 + PIP | .926 | 1.0 | .958 | .915 | 1.0 | .953 | .005 |
| MGB2 + TACSTD1 | .963 | 1.0 | .979 | .930 | 1.0 | .961 | .018 |
| PIP + TACSTD1 | .963 | 1.0 | .979 | .929 | 1.0 | .960 | .017 |
| **500 parametric bootstrap samples of 48 lymph node expression levels (27 positive, 21 benign were generated from the bivariate log-normal distribution and a new decision rule and a new decision rule formulated each time. The differences between classifying the original data and classifying the bootstrap data were averaged to form the estimate of bias in the re-substitution decision rule. The respective estimated bias was then subtracted from the | | | | | | | |
| # sensitivity, specificity and classification accuracy of the original data to arrive at the bootstrap estimates. The bias in the estimated classification accuracy is shown in the last column. | | | | | | | |

**Classification Bias = average difference in classification accuracies of the bootstrap decision rule applied to original data and the bootstrap decision rule applied to the bootstrap data. This estimates the optimism in using the original data to characterize the decision rule.

### Example 3

### Follow-On Study—Breast Cancer Materials and Methods

As outlined above in Example 2, an extensive literature and database survey identified potential mRNA markers for detection of lymph node metastases in breast cancer. A primary screen analyzed the relative expression of 43 potential markers in 6 primary breast tumors and 10 benign lymph nodes obtained from patients without cancer. Six markers showed good characteristics for lymph node metastasis detection and entered a secondary screening phase where expression was analyzed in 25 primary tumors, 27 histologically positive lymph nodes and 21 benign lymph nodes from patients without cancer (73 independent patients). Based on the classification characteristics, 4 markers were selected for an external validation study of 90 SLN from independent patients with breast cancer using a rapid, multiplex real-time PCR assay. Finally, 9 histologically negative and 9 histologically positive lymph nodes were analyzed using a completely automated and rapid RNA isolation and real-time PCR assay on the GeneXpert®.

Source of Tissues. Tissues for the marker screening and the GeneXpert® study were obtained from tissue banks at the University of Pittsburgh Medical Center and SLN for the marker validation study were obtained from the Minimally Invasive Molecular Staging of Breast Cancer Trial (MIMS) initiated at the Medical University of South Carolina.

Tissue Preparation and Histologic Analysis. All tissues were snap-frozen in liquid nitrogen and stored at −80° C. until use, at which time they were embedded in optimal cutting temperature (OCT) compound for frozen sectioning on a cryostat. For the marker screening and GeneXpert® studies, forty 5-micron sections were cut for RNA isolation. Additional sections from the beginning, middle and end of the sections for RNA isolation were cut for H&E and IHC analysis. All three H&E slides from each specimen underwent pathological review by two pathologists. All unstained slides were stored at −20° C. and used for IHC evaluation (with the AE1/AE3 pancytokeratin antibody cocktail) as needed to confirm the H&E histology.

For the validation study, 115 chronologically-obtained SLN specimens from individual patients were identified. Five-micron serial sections were cut from each tissue, and the initial and final two tissue sections were mounted on slides for histological analysis with H&E staining and pancytokeratin IHC. The intervening sections were distributed 4:1:4:1:4 etc., such that four sections were immediately placed in chaotropic lysis buffer for RNA isolation and every fifth section was mounted on a slide for histology review. The total number of sections cut was dependent on size of the SLN (range 50-60). All specimens were reviewed to confirm adequate preservation of histology for pathologic analysis resulting in the exclusion of 25 specimens. For the remaining 90 SLN, sections from three levels (beginning, middle and end) underwent pathologic review with both H&E and IHC staining, and remaining slides were reviewed as needed.

All specimens were independently evaluated by two pathologists with extensive experience interpreting breast cancer specimens. The pathologists determined the presence of tumor, the percentage of tumor, and the presence of any contaminating tissues (e.g., normal breast tissue). Discordantly interpreted specimens were noted, and then reviewed simultaneously and consensus made.

RNA Isolation. For the screening and validation studies, RNA was isolated using the RNeasy mini kit (Qiagen, Valencia, Calif.) as described by the manufacturer. The only modification was that the volume of lysis reagent was doubled and loaded on the column in two steps. All RNA's were DNAse treated using the DNA-free Kit from Ambion.

Quantitative RT-PCR Analysis. For the marker screening study, cDNA was synthesized using random hexamers. Quantitative real-time PCR was performed on the ABI Prism 7700 Sequence Detection instrument and expression of each marker gene was measured relative to the endogenous control gene β-glucuronidase using ΔCt calculations. To save cost, the primary screen was performed using quantification with SYBR green. In the secondary screen, 5′ nuclease hybridization probes were used to increase assay specificity. All assays were designed using the ABI Primer Express Version 2.0 software and where possible, amplicons spanned exon junctions to provide cDNA specificity. Negative controls were included in each PCR plate. A mixture of the Universal Human Reference RNA (Stratagene, La Jolla, Calif.) and RNAs from human placenta, thyroid, heart, colon, PCI13 cell line and SKBR3 cell line served as a universal positive expression control for all the genes in the marker screening process.

Analysis of the four genes in the marker validation study was performed using rapid, multiplex (endogenous control gene and target gene) QRT-PCR on the Cepheid SmartCycler® (Cepheid, Sunnyvale, Calif.). RNA input for each lymph node sample was 50-200 ng per QRT-PCR reaction and all reactions were performed in duplicate. Each reaction incorporated an internal positive control (IPC) oligonucleotide to demonstrate adequate assay sensitivity in the case of negative results. Gene specific reverse transcription primer sequences and PCR primer and probe sequences are shown in Table B.

GeneXpert® Analysis. Twenty-four, 5-μm sections of OCT embedded tissue were sectioned into 800 μL of GeneXpert® lysis buffer (Cepheid, Sunnyvale, Calif.). The lysis buffer was filtered through a 0.22 μm syringe filter (Osmonics Inc, Westborough, Mass.), and loaded into a GeneXpert® cartridge. The automated processes of RNA isolation, reverse transcription, and QRT-PCR on the GeneXpert®.

Statistical Analyses. The characteristics used to evaluate markers were sensitivity, specificity, classification accuracy and negative and positive predictive values. The evaluation included characterizing the distributions of the markers and testing the fit of the data to the log-normal distribution. For individual markers, a cutoff value was determined that maximized the classification accuracy. In cases where classification accuracy was 100%, the cutoff was set at the midpoint between the highest expressing benign node and the lowest expressing histologically positive node. Markers were also evaluated in paired combinations and a linear prediction rule was generated for each pair. The rule was equivalent to the linear predictor that equalized the fitted probabilities above and below the linear boundary. That is, points on the boundary line had a predicted probability midway between the numeric scores assigned to positive and negative nodes.

Properties of single and paired marker prediction rules were also investigated by examining the distributional properties of the expression levels and by applying parametric bootstrap validation. Data were simulated from the log normal and bivariate lognormal distributions using moment estimators for mean, variance and correlation between marker pairs. Five hundred parametric samples of the original data were obtained and the prediction for each bootstrap sample was applied to the original data. Using Efron's improved bootstrap for prediction error (Efron B, TR. An Introduction to the Bootstrap. Boca Raton: Chapman and Hall, 1993: 247-252), the difference between the observed classification accuracy and the average bootstrap classification accuracy was used to estimate the optimism in the resubstitution prediction rules. The single marker and double marker decision rules were then applied to data from the marker validation study and classification characteristics were calculated.

Prediction characteristics of marker combinations were also determined by generating equal probability contours. In this method, the joint distributions of marker pairs were assumed to follow a bivariate log-normal distribution. From estimates of the means, variances and covariances of benign nodes, equal probability contours were constructed around the estimated mean values obtained for relative level of expression in benign lymph nodes. Observed values were then plotted against these equal probability ellipsoids and compared to contours for the more extreme quantiles, including the 95^{th}, 99^{th} and 99.9^{th} percentiles. This method of analyzing the data attaches a value to each point that is the approximate probability that the plotted node is benign.

Results

Primary Marker Screen. Median relative expression in primary tumors and in benign lymph nodes was calculated for all 43 potential markers included in the primary screen (Table F).

TABLE F
relative expression in primary tumor and benign lymph node from primary screening

| | | | Median | Highest | Lowest | Median |
|---|---|---|---|---|---|---|
| | GenBank | Median | Benign | Benign | Tumor/Highest | Tumor/Highest |
| Gene | Accession No. | Tumor | Node | Node | Benign Node | Benign Node |
| TACSTD1 | NM_004616 | 56.797758 | 0.009753 | 0.049549 | 643.6 | 1146.3 |
| CK19 | NM_002276 | 34.646797 | 0.003691 | 0.018136 | 1086.1 | 1910.4 |
| CK7 | NM_005556 | 14.725973 | 0.000953 | 0.003162 | 22.2 | 4657.3 |
| CK18 | NM_000224 | 6.956115 | 0.033609 | 0.073557 | 18.1 | 94.6 |
| MMP7 | NM_002423 | 0.626631 | 0.009453 | 0.099098 | 1.6 | 6.3 |
| MGB1 | NM_002411 | 0.283272 | 0.000034 | 0.000207 | 10.1 | 1365.5 |
| Survivin | NM_003317 | 0.186357 | 0.022251 | 0.113834 | 0.1 | 1.6 |
| PIP | NM_002652 | 0.117351 | 0.000519 | 0.001258 | 1.0 | 93.3 |
| MGB2 | NM_002407 | 0.106771 | c | c | ∞ | ∞ |
| c-MET | NM_000245 | 0.061745 | 0.020617 | 0.039692 | 0.3 | 1.6 |
| PTHrP | NM_002820 | 0.024784 | 0.001186 | 0.004425 | 2.5 | 5.6 |
| NIS | NM_000453 | 0.015681 | 0.003933 | 0.016232 | 0.02 | 1.0 |
| TM4SF3 | NM_004616 | 0.013634 | 0.025295 | 0.136787 | 0.01 | 0.1 |
| BHCG | NM_000737 | 0.007882 | 0.001893 | 0.009005 | 0.4 | 0.9 |
| CEA | NM_004363 | 0.007112 | 0.000175 | 0.000270 | 1.3 | 26J |
| SCCA1/2 | NM_006919 | 0.005290 | 0.000014 | 0.015571 | 0.04 | 0.3 |
| MAGEA8 | NM_005364 | 0.004843 | c | c | ∞ | ∞ |
| Villin 1 | NM_007127 | 0.003967 | 0.000288 | 0.000433 | 0.8 | 9.2 |
| KRTHB1 | NM_002281 | 0.002590 | b | 0.000165 | 4.2 | 15.7 |
| T1TF1 | NM_007127 | 0.001900 | 0.001320 | 0.007625 | 0.03 | 0.2 |
| HTERT | NM_003219 | 0.001455 | 0.012648 | 0.026645 | 0.01 | 0.1 |
| ITGB4 | NM_000213 | 0.000799 | 0.000038 | 0.000063 | 3.2 | 12.6 |
| STX | NM_002354 | 0.000728 | 0.000030 | 0.000194 | 0.8 | 3.7 |
| LDHC | NM_017448 | 0.000487 | b | 0.016402 | 0.00006 | 0.03 |
| BAGE | NM_001187 | 0.000445 | b | 0.000152 | 0.007 | 2.9 |
| CTAG1 | NM_001327 | 0.000416 | b | 0.002036 | 0.0005 | 0.2 |
| NTS | NM_006183 | 0.000305 | 0.532185 | 2.321408 | 0.0 | 0.001 |
| MAGEA2 | NM_005361 | 0.000183 | b | 0.000279 | 0.004 | 0.7 |
| CK20 | NM_019010 | 0.000161 | c | c | ∞ | ∞ |
| GAGE1 | NM_001468 | 0.000144 | b | 0.000703 | 0.001 | 0.2 |
| SSX2 | NM_006011 | 0.000136 | c | c | ∞ | ∞ |
| MAGEA3 | NM_005362 | 0.000116 | 0.000328 | 0.001271 | 0.001 | 0.1 |
| SSXu^{a} | NM_001169 | 0.000061 | c | c | ∞ | ∞ |
| BRDT | NM_001726 | 0.000037 | 0.000278 | 0.000350 | 0.003 | 0.1 |
| SGY-1 | NM_014419 | 0.000024 | 0.000134 | 0.000605 | 0.002 | 0.04 |
| GAGEu^{a} | | 0.000023 | 0.000010 | 0.000126 | 0.1 | 0.2 |
| MAGEA12 | NM_005367 | 0.000022 | 0.000103 | 0.000404 | 0.002 | 0.1 |
| MAGEA1 | NM_004988 | 0.000017 | c | c | ∞ | ∞ |
| MAGEA4 | NM_002362 | 0.000011 | c | c | ∞ | ∞ |
| CK14 | NM_000526 | c | c | c | ∞ | ∞ |
| LUNX | NM_130852 | b | c | c | ∞ | ∞ |
| MAGEA10 | NM_021048 | c | c | c | ∞ | ∞ |
| TYR | NM_000372 | b | c | c | ∞ | ∞ |

In addition, the ratio was calculated between the median expression in tumors and the highest expressing benign node and between the lowest expressing tumor and the highest expressing benign node. When using median expression in the tumors as the numerator, four genes, TACSTD1, cytokeratin 7 (CK7), cytokeratin 19 (CK19), and mammoglobin 1 (MGB1) stood out as having tumor/benign node ratios greater than 1000. Thus, these 4 markers were selected for further evaluation. Mammoglobin 2 (MGB2) and prolactin inducible protein (PIP) were also selected based on the primary screen data as well as previously published data regarding these markers (Mitas M, et al., “Quantitative real-time RT-PCR detection of breast cancer micro metastasis using a multigene marker panel”, Int J Cancer 2001; 93(2): 162-171). The other 37 markers were excluded from further evaluation.

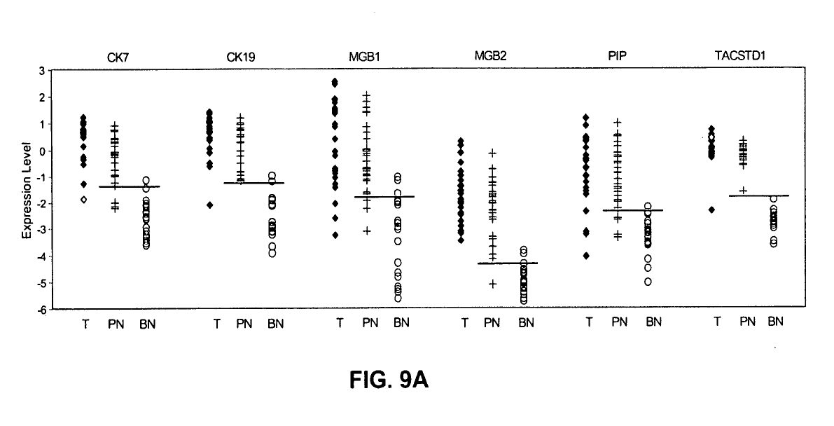

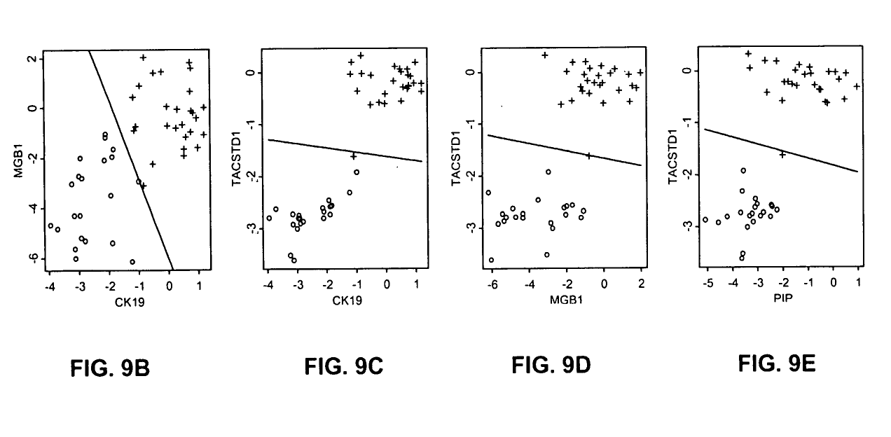

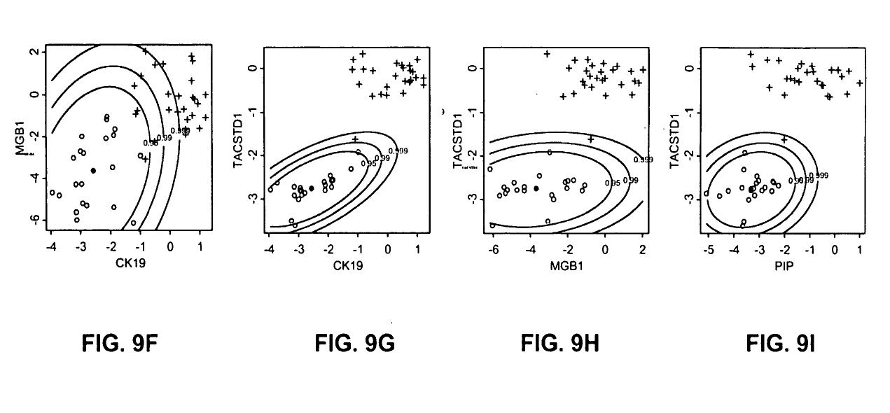

Secondary Marker Screen. Histologic evaluation of the 25 primary breast cancer specimens used in the secondary screen revealed a median tumor percentage of 75% (range of 5-95%). The median tumor percentage in the 27 histologically positive nodes was 80% (range of 5-95%). The relative expression of the 6 markers included in the secondary screen in breast tumors, positive lymph nodes, and benign lymph nodes are shown in FIG. 9A. The classification characteristics of each marker (compared with pathology review) are summarized in Table G.

TABLE G
Single or two marker prediction characteristics in secondary screening

| | Observed Data | | | Parametric Bootstrap Estimates* | | | |
|---|---|---|---|---|---|---|---|
| | | | Classification | | | Classification | Classification |
| Marker | Sensitivity | Specificity | Accuracy | Sensitivity | Specificity | Accuracy | Bias** |
| CK7 | .889 | .952 | .917 | .828 | .909 | .863 | .054 |
| CK19 | 1.0 | .952 | .979 | .997 | .891 | .951 | .028 |
| MGB1 | .926 | .857 | .896 | .903 | .748 | .836 | .060 |
| MGB2 | .963 | .905 | .938 | .943 | .834 | .895 | .043 |
| PIP | .852 | .952 | .896 | .814 | .892 | .848 | .048 |
| TACSTD1 | 1.0 | 1.0 | 1.0 | .999 | .956 | .980 | .020 |
| CK7 + CK19 | 1.0 | .905 | .958 | .993 | .868 | .938 | .020 |
| CK7 + MGB1 | .963 | 1.0 | .979 | .954 | 1.0 | .977 | .002 |
| CK7 + MGB2 | 1.0 | 1.0 | 1.0 | 1.0 | 1.0 | 1.0 | .000 |
| CK7 + PIP | .963 | 1.0 | .979 | .963 | 1.0 | .979 | .000 |
| CK7 + TACSTD1 | .963 | 1.0 | .979 | .928 | 1.0 | .959 | .020 |
| CK19 + MGB1 | 1.0 | 1.0 | 1.0 | .996 | 1.0 | 1.0 | .000 |
| CK19 + MGB2 | .963 | 1.0 | .979 | .945 | .979 | .975 | .004 |
| CK19 + PIP | .926 | 1.0 | .958 | .900 | 1.0 | .951 | .007 |
| CK19 + TACSTD1 | .963 | 1.0 | .979 | .928 | 1.0 | .960 | .019 |
| MGB1 + MGB2 | .889 | .952 | .917 | .853 | .925 | .885 | .032 |
| MGB1 + PIP | .963 | .905 | .938 | .963 | .876 | .934 | .004 |
| MGB1 + TACSTD1 | .963 | 1.0 | .979 | .942 | 1.0 | .967 | .012 |
| MGB2 + PIP | .926 | 1.0 | .958 | .915 | 1.0 | .953 | .005 |
| MGB2 + TACSTD1 | .963 | 1.0 | .979 | .930 | 1.0 | .961 | .018 |
| PIP + TACSTD1 | .963 | 1.0 | .979 | .929 | 1.0 | .960 | .017 |

The observed classification accuracies ranged from 89.6% (MGB1 and PIP) to 100% (TACSTD1). Parametric bootstrap analysis of this data is also shown in Table G and the estimates of classification bias ranged from 2% (TACSTD1) to 6% (MGB1). Thus, the relative expression level cut-offs established for each individual marker in the screening set should accurately characterize subsequently analyzed lymph nodes.

All possible combinations of marker pairs were examined to determine if an assay that evaluates more than one marker produces a more robust lymph node characterization. The relative expression of each possible marker pairing was analyzed using a linear decision rule that optimized characterization accuracy and these decision rules were again internally validated using a parametric bootstrap analysis. This data is illustrated in FIGS. 9B-9E and summarized in Table G. Eleven of the 15 combinations provided 100% classification accuracy in the observed data but only two combinations retained 100% predicted accuracy in the bootstrap analysis. In general, the use of a pair of markers resulted in a reduction in classification bias (0-3.2%) confirming that a 2-marker assay improved assay classification confidence.

Since linear classification rules are not necessarily the best method for lymph node classification in the marker combination analysis, a novel classification method was developed based on the observed distribution of expression levels for each marker in a given pair. Equal probability contours were calculated around the mean values obtained for relative expression in benign lymph nodes (for example, see FIGS. 9F-9I). This method of analysis demonstrates that the distribution of relative expression values obtained from benign lymph nodes impacts the confidence for classifying a positive lymph node. While CK19/MGB1 provided the best classification based on a linear prediction rule, the probability contour plot clearly shows that the wide distribution of expression for both of these markers in benign nodes negatively impacts the confidence with which a positive node can be identified. By this analysis, the combinations of TACSTD1/PIP, CK19/TACSTD1, TACSTD1/MGB1 and TACSTD1/MGB2 provide the best classification with all positive nodes correctly identified with probabilities >0.99 and in most cases >0.999. For all four of these combinations, all benign nodes fell within the 0.99 probability contour and all but one was within the 0.95 probability contour. Therefore, in the screening data, four marker combinations were capable of providing 100% sensitivity with >99% specificity.

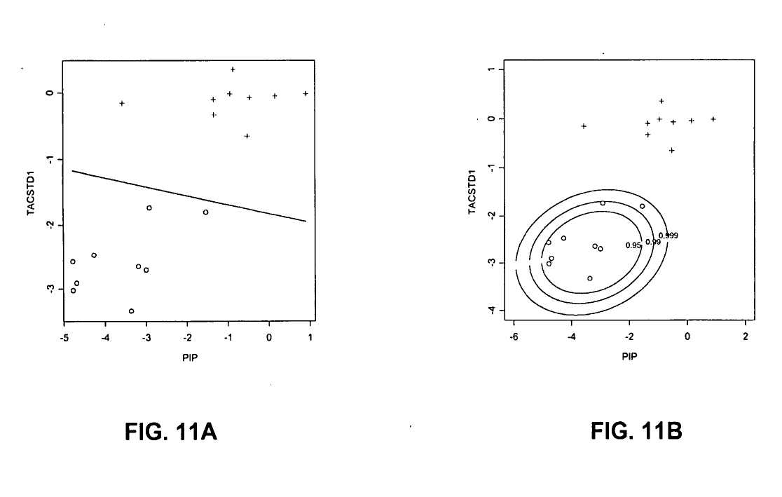

Validation of QRT-PCR classification in a Rapid, Multiplex format. To externally validate the classification accuracy of selected markers tested in the secondary screen, an independent, validation set of 90 breast cancer sentinel lymph nodes was prospectively analyzed (FIGS. 11A and 11B). Furthermore, to demonstrate the potential for intraoperative analysis, this study was performed on the SmartCycler® instrument (Cepheid) using rapid, multiplex QRT-PCR. Subtle differences in calculated relative expression values were observed (data not shown) from this change in thermocycler platform, but in an effort to indirectly evaluate the robustness of the QRT-PCR analysis, the classification algorithms from the secondary screen were applied to the validation set data without any correction factors.

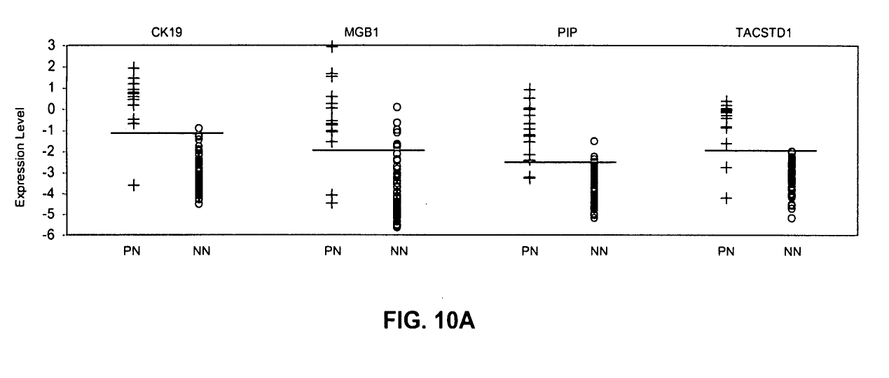

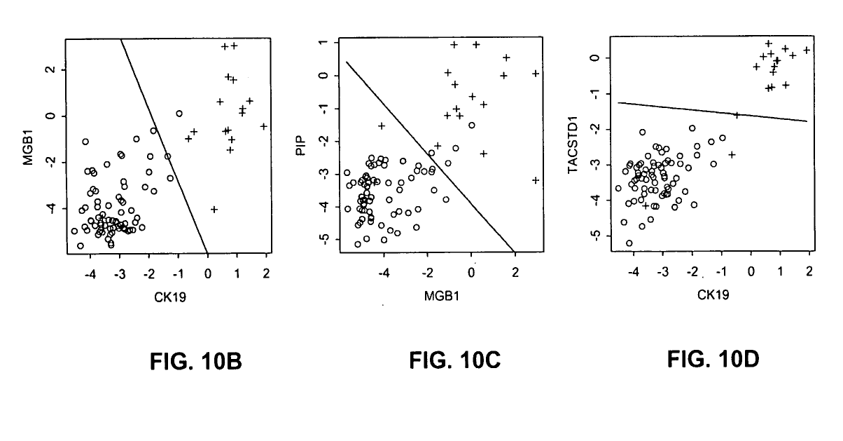

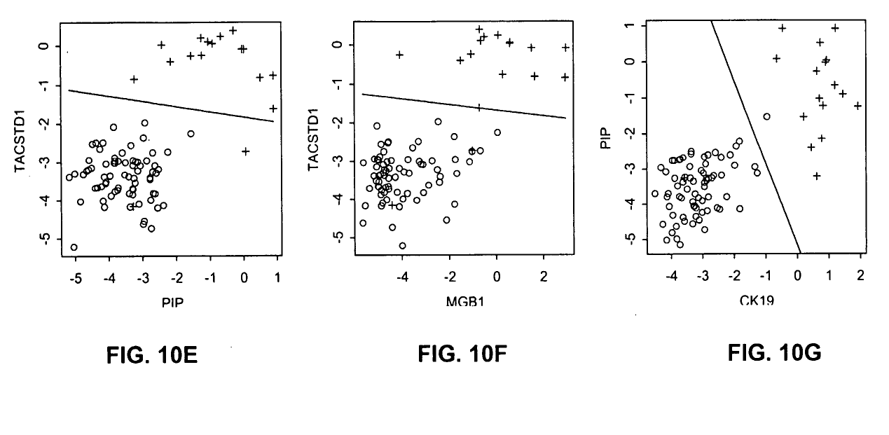

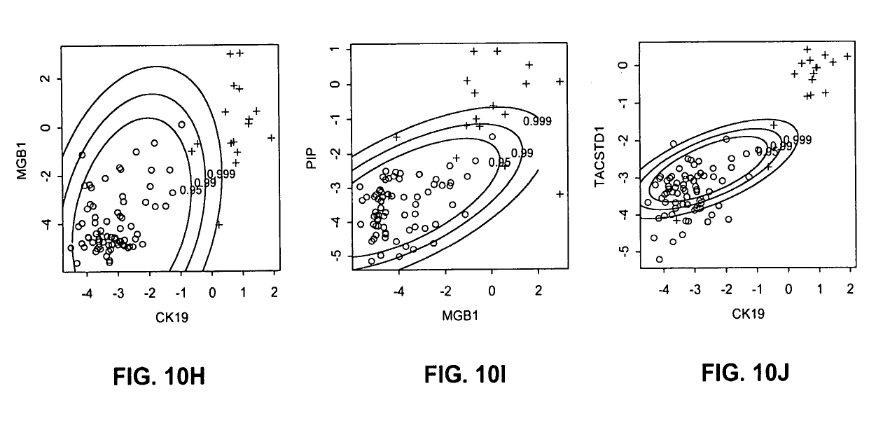

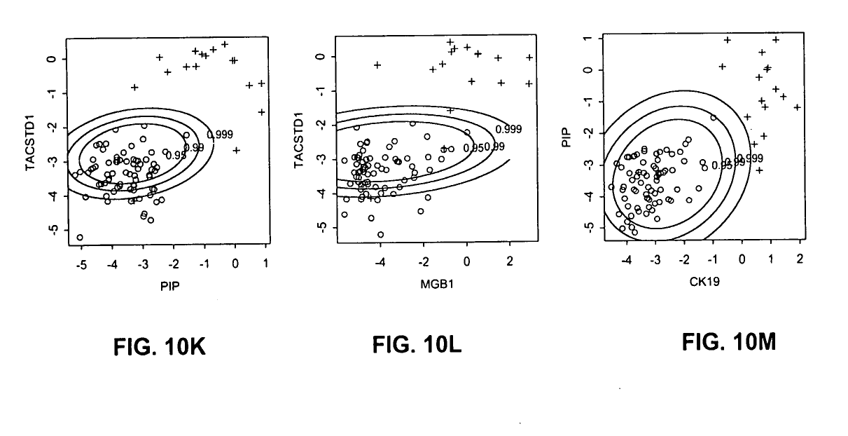

Pathologic review identified 73 negative SLN's and 17 SLN's positive for metastasis, with a median tumor percentage in the positive lymph nodes of 60% (range 5%-95%). The relative expression data for each of the 4 selected markers, and marker combinations, is shown in FIGS. 10A-10M, and prospective classification accuracy for individual markers and all potential marker pairs is reported in Table H.

TABLE H
Validation set results. Prospective classification characteristics
of QRT-PCR assays using single or paired markers

| Marker/ | | | | | |
|---|---|---|---|---|---|
| Combination | Sensitivity | Specificity | Accuracy | NPV* | PPV** |
| PIP | .882 | .959 | .944 | .972 | .833 |
| MGB1 | .882 | .890 | .889 | .970 | .652 |
| TACSTD1 | .882 | 1.0 | .978 | .973 | 1.0 |
| CK19 | .941 | .986 | .941 | .978 | .986 |
| PIP + MGB1 | .882 | .944 | .933 | .971 | .789 |
| PIP + TACSTD1 | .882 | 1.0 | .978 | .973 | 1.0 |
| PIP + CK19 | .941 | .986 | .978 | .986 | .941 |
| MGB1 + | .823 | 1.0 | .966 | .960 | 1.0 |
| TACSTD1 | | | | | |
| MGB1 + CK19 | .941 | .986 | .978 | .986 | .941 |
| TACSTD1 + | .823 | 1.0 | .966 | .960 | 1.0 |
| CK19 | | | | | |

*NPV = negative predictive value;
**PPV = positive predictive value.

When cut-off values (individual markers) or linear prediction rules (marker combinations) from the secondary screen were applied to the validation set data, overall classification accuracy ranged from 89% (MGB1 alone) to 98% (TACSTD1 alone, TACSTD1/PIP, PIP/CK19 and MGB1/CK19). When probability contours from the secondary screen were applied, several marker combinations identified 16/17 (94%) of positive nodes with >99.9% probability while all negative nodes fell within the 99% probability contour. One histologically positive sample was characterized as negative with >95% probability by analysis with all 4 markers. In a post-analysis review, this specimen was found to have been discordantly interpreted by the two pathologists. A concurrent opinion had been reached, based on a very small focus of tumor in the first 2 serial sections that was not present in the remaining 8 slides. Thus, our finding that this specimen was consistently classified as negative by QRT-PCR may represent sampling error.

From these data, it was concluded that there are a number of mRNA markers and marker combinations capable of accurately detecting metastatic breast cancer in lymph nodes. However, there are at least 3 pseudo genes for CK19 within the human genome that lack intronic sequence. Thus, an mRNA-specific primer set cannot be designed for CK19, and failure of DNAse treatment to completely digest contaminating genomic DNA within the sample could produce a false positive result. Thus, the combination that produces the highest accuracy without other potentially negative attributes is the marker pair of TACSTD1 and PIP.

Automated Lymph Node Analysis with the GeneXpert®. Eighteen lymph node specimens from individual patients were evaluated with fully automated, QRT-PCR assays for the markers TACSTD1 and PIP (FIGS. 11A and 11B). Histologic review confirmed that this set consisted of 9 positive (60-95% tumor) and 9 negative lymph nodes. When prospectively analyzed by either a linear decision rule or equal probability contour analysis using decision rules based on data from the secondary screen set, the multiplex GeneXpert® assay accurately (100%) characterized all 18 specimens within 35 minutes per assay. It is concluded that a fully automated, rapid QRT-PCR assay accurately characterizes lymph nodes for the presence of metastatic breast cancer.

The above-described methods are seen to provide exceptional accuracy detecting metastatic disease within the SLN of breast cancer patients using a two-marker QRT-PCR assay compared to the current methods of complete SLN analysis including histological and immunohistochemical review. Also demonstrated is the accurate classification of the lymph node specimens obtained when the assay was fully automated using the GeneXpert® instrument. Thus, this assay surpasses the accuracy of current frozen section analysis of SLNB specimens, and is potentially superior to complete histological and IHC analysis in that: 1) it is fully automated, reducing the potential for human error, 2) it uses objective criteria, removing subjective analysis and improving standardization, and 3) it is completed in less than 35 minutes, facilitating intraoperative use and reducing anxious apprehension for the patient.

Previous studies have aimed to determine if RT-PCR analysis of lymph nodes is more sensitive than IHC and thus capable of further improving the clinical staging of breast cancer patients. The present study differs from those studies in that the present aim was not to determine if QRT-PCR identified metastatic disease in definitively analyzed, histologically negative SLN, but rather to surpass current methods of analysis with regards to timeliness, reproducible objectivity, and automation. However, based on the published literature regarding sensitivity of QRT-PCR analyses and the ability of this automated assay to improve sampling by evaluating a larger percentage of the LN (current SLNB analysis examines less than 1.5% of the specimen), it is believed that this assay may prove to be capable of surpassing current techniques in this regard.

This assay ultimately may prove to be superior to conventional histological analysis because of the objective nature of the test, but this benefit is implied and has not yet been scientifically proven. The accurate histological analysis of lymph nodes for micrometastatic disease is challenging under ideal conditions, by nature subjective, and the interpretation of microscopic foci of tumor cells has eclipsed clinical outcome data. The AJCC Cancer Staging Manual, 6th edition has established definitions to facilitate consistency in interpretation of these materials, yet these definitions make further demands on the pathologist's subjective interpretation of the lymph node. In the only published study examining this problem, Roberts, et al. found that when 10 pathologists evaluated 25 cases of breast cancer SLNB specimens, only 12% of the cases were correctly classified by all the pathologists, and 80% of the IHC-positive cases had at least one pathologist incorrectly characterize the case (Roberts C A, et al., “Interpretive disparity among pathologists in breast sentinel lymph node evaluation,” Am. J. Surg. 2003; 186(4):324-329). In contrast, as demonstrate herein and separately, the fully automated QRT-PCR assay is robust and objective. Thus, a reproducible, fully automated, objective analysis of SLNs has the potential to be superior to current methods of analysis, and a multi-center, prospective trial designed to make this comparison is currently in development.

In summary, it has been shown that a two-marker, QRT-PCR assay that is fully automated and completed in under 35 minutes can accurately characterize lymph nodes for the presence of metastatic breast cancer. This assay is clearly superior to current methods of intraoperative analysis and is as accurate as current methods of complete histological analysis including immunohistochemical analysis. Theoretical advantages to such an assay include improved standardization across varying healthcare environments, increased sampling of the lymph node, and reduced human error.

### Example 4

### Three-Marker Multiplex PCR Assay

Primer and Probe Design. All three primer/probe sets were designed to span an exon junction to reduce the probability of amplifying genomic DNA and in a region of low overall homology to other known genes. Primer pairs for the two target genes were designed to generate amplicons of at least 4° C. higher denaturation temperature than that of the endogenous control gene. Initially, the primer pairs for the two target genes were designed to have similar anneal temperatures to the endogenous control gene. The primers for the target genes were then modified by the addition of a “GC clamp” to raise the anneal temperature by at least 4° C. over the endogenous control (Table I).

TABLE I

| | | | | | | |
|---|---|---|---|---|---|---|
| | | | | | | Predicted |
| | Primer | | Sequence | Anneal | Predicted | Percent |
| Gene | or Probe | Sequence | Reference | Temp. | Tm | Binding |

| | | | | | | |
|---|---|---|---|---|---|---|
| PIP | Forward | GCGGCTCCAGCTCCTGTTCAG | SEQ ID NO: 17 | 68° C. | 73° C. | 97.7 |
| | Reverse | GGCGCATTATGATCTTCCGAGTGTTGTC | SEQ ID NO: 18 | 68° C. | 72° C. | 98.8 |
| | Probe | CCAGCCCTGCCACCCTGCTCCTG; | SEQ ID NO: 5 | 68° C. | 74° C. | 100 |
| | | | bases 65 to 87 | | | |
| | RT | GGGAATGTCAAAATTCTTT | SEQ ID NO: 19 | | 48° C. | |
| TACSTD1 | Forward | GCGCGTTCGGGCTTCTGCTT | SEQ ID NO: 20 | 68° C. | 72° C. | 97.3 |
| | Reverse | GCGAGTTTTCACAGACACATTCTTCCTGAG | SEQ ID NO: 21 | 68° C. | 70° C. | 95.8 |
| | Probe | CGCGGCGACGGCGACTTTTGC; | SEQ ID NO: 6 | 68° C. | 74° C. | 100 |
| | | | bases 220 to 240 | | | |
| | RT | AGTTTACGGCCAGCTTG | SEQ ID NO: 22 | | 48° C. | |
| β-GUS | Forward | CTCATTTGGAATTTTGCCGAT | SEQ ID NO: 23 | 60° C. | 63° C. | 92.5 |
| | Reverse | CCGAGTGAAGATCCCCTT | SEQ ID NO: 24 | 60° C. | 64° C. | 93.6 |
| | Probe | TGAACAGTCACCGACGAGAGTGCTGG | SEQ ID NO: 25 | 68° C. | 73° C. | 100 |
| | RT | TTTGTTGTCTCTGCCGAGT | SEQ ID NO: 26 | | 48° C. | |

The primer and probe sequences are shown for the endogenous control gene, β-GUS and for the two target genes, PIP and TACSTD1. Also shown are the anneal temperature conditions under which software simulations using Visual OMP software were run to predict primer/probe melting temperatures and percent binding.

Anneal Temperature Studies. Anneal temperature studies were performed in single-plex on the SmartCycler® (Cepheid) platform in 25 μL reaction tubes. The plasmids pENTR-TACSTD1, pENTR-PIP, and pOT7-βGUS were obtained from Invitrogen and used at approximately 4E3 and 3E6 copies per reaction for low copy and high copy respectively. All probes were used at 200 nM. Primers for PIP and TACSTD1 were used at 400 nM. Primers for β-GUS were used at 600 nM. Reactions were carried out by 2.5U Platinum Taq in manufacturer's buffer supplemented with 4 mM MgCl₂ and 200 μM dNTPs. Cycling conditions for β-GUS amplification were conducted using a 2-stage protocol. In Stage 1, the samples were denatured for 30 seconds at 95° C. In Stage 2, a 40-cycle 3-temperature protocol was used consisting of a denaturation step at 95° C. for 1 second, an anneal step that was varied from 56° C. to 68° C. for 4 seconds, and an optical read/extension step at 68° C. for 6 seconds.

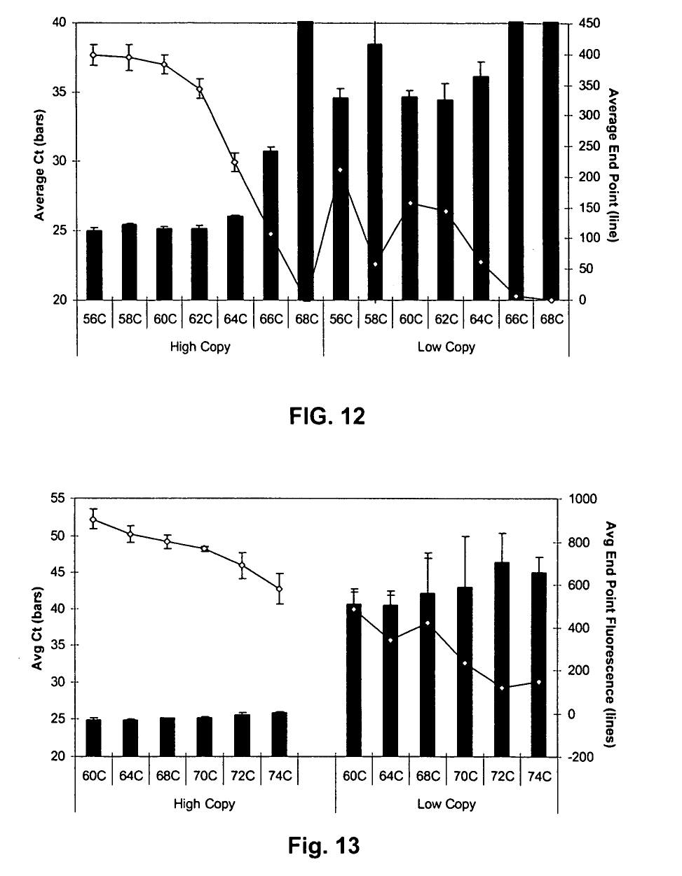

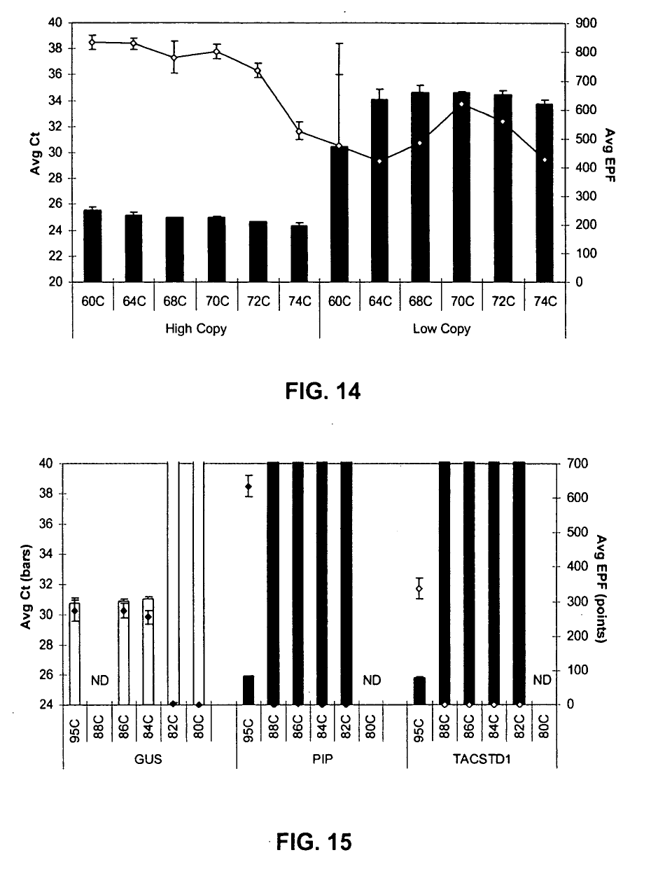

Cycling conditions for PIP and TACSTD1 were conducted using a 3-stage protocol. In Stage 1, the samples were denatured for 30 seconds at 95° C. In Stage 2, a 20-cycle 3-temperature protocol was used consisting of a denaturation step at 95° C. for 1 second, an anneal step at 60° C. for 4 seconds, and an optical read/extension step at 68° C. for 6 seconds. In Stage 3, a 30-cycle 2-temperature protocol was used consisting of a denaturation step at 95° C. for 1 second and an anneal/extension/optical read step that was varied from 60° C. to 74° C. for 6 seconds. FIGS. 12, 13 and 14 show the affect of anneal temperature on PCR performance for the β-GUS, PIP and TACSTD1 assays. FIG. 12 shows β-GUS amplification performance is non-optimal above 62° C. and fails completely at 68° C. FIGS. 13 and 14 show that both PIP and TACSTD1 amplify optimally at least to 74° C. anneal temperature.

Serial Dilution Method for Efficiency Studies. For simplex studies, plasmids were serially diluted in TE buffer from 3000 pg to 0.092 pg and used as template in the PCR reaction. Reactions were carried out on the SmartCycler® platform using 25 μL reaction tubes. Primer concentrations, probe concentrations, enzyme, MgCl₂, and dNTP concentrations were the same as those used for the anneal temperature studies.

For PIP and TACSTD1, the cycling protocol was a 3-stage protocol consisting of: Stage 1, 95° C. for 30 seconds for 1 cycle; Stage 2, 95° C. for 1 second, 60° C. for 4 seconds, and 68° C. for 6 seconds with optics “on” for 20 cycles; and Stage 3, 95° C. for 1 second and 68° C. for 6 seconds with optics “on” for 20 cycles.

For β-GUS, the cycling protocol was a 3-stage protocol consisting of: Stage 1, 95° C. for 30 seconds for 1 cycle; Stage 2, 95° C. for 1 second, 60° C. for 4 seconds, and 68° C. for 6 seconds with optics “on” for 20 cycles; and Stage 3, 86° C. for 1 second, 60° C. for 4 seconds, and 68° C. for 6 seconds with optics “on” for 20 cycles.

For triplex efficiency studies, duplicate samples were prepared for each point and subjected to each cycling protocol independently. Template mixtures were prepared by serially diluting each plasmid separately and mixing them in various ratios. Reaction conditions and volumes were the same as for simplex reactions.

The expected amplicon sizes and denaturation temperatures are shown in Table J below along with the reaction efficiencies measured in simplex and in triplex using the serial dilution method.

TABLE J

| | Anneal | | | | Denaturation |
|---|---|---|---|---|---|
| | Temperature | | | Amplicon | Temperature |
| | (° C.) | Gene | Efficiency | Size | (° C.) |
| Simplex | 60/68 | PIP | 100 | 110 | 88 |
| | 60/68 | TACSTD1 | 96.6 | 79 | 84 |
| | 60 | β-GUS | 99.5 | 81 | 78 |
| Triplex | 60/68 | PIP | 95.3 | 110 | 88 |
| | 60/68 | TACSTD1 | 97.2 | 79 | 84 |
| | 60 | β-GUS | 94.5 | 81 | 78 |

Denaturation Temperature Study. Denaturation temperature studies were carried out in simplex reactions set up as described above. The cycling protocol used consisted of a 3-stage protocol: Stage 1, 95° C. for 30 seconds for 1 cycle; Stage 2, 95° C. for 1 second, 60° C. for 4 seconds, and 68° C. for 6 seconds with optics “on” for 20 cycles; and Stage 3, various temperatures from 80° C. to 95° C. for 1 second, 60° C. for 4 seconds, and 68° C. for 6 seconds with optics “on” for 20 cycles. FIG. 15 shows that β-GUS amplification proceeds normally with a denaturation temperature as low as 84° C., but PIP and TACSTD1 reactions fail at 88° C. or lower.

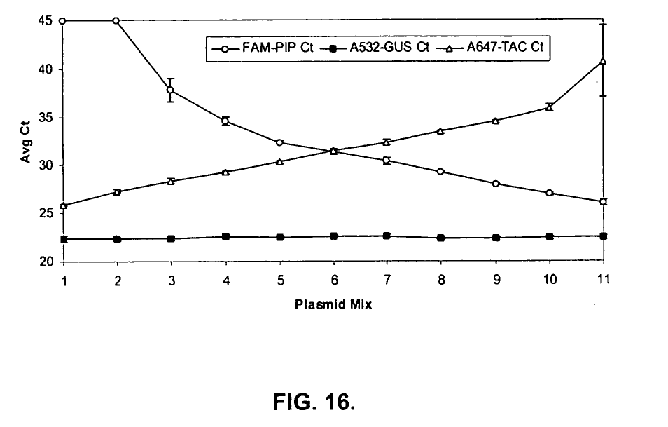

Dynamic Range of Targets in the Presence of High endogenous control. Plasmid mixes of various ratios were prepared as shown in Table K. Triplex reactions were set up in triplicate as described above. The cycling protocol was a 3-stage protocol consisting of: Stage 1; 95° C. for 30 seconds for 1 cycle; Stage 2, 95° C. for 1 second, 60° C. for 4 seconds, and 68° C. for 6 seconds with optics “on” for 20 cycles; and Stage 3, 95° C. for 1 second and 68° C. for 6 seconds with optics “on” for 30 cycles. The average and standard deviations of the Ct values obtained are shown in FIG. 16. These data demonstrate that the dynamic range of the two target genes (PIP and TACSTD1) is linear with high efficiency with a fold difference in expression of at least 6 Cts.

TABLE K

| | | | | Stock | | | | |
|---|---|---|---|---|---|---|---|---|
| | | | | Conc. | Dilution | | | |
| | | Size (bp) | Est FW | (ng/μL) | Factor | g/rxn | Mol/rxn | copies/rxn |
| Mix 1 | pENTR-PIP | 2987 | 1822070 | 232 | 3.14E+08 | 3.69E−15 | 2.03E−21 | 1.22E+03 |
| | pENTR-TACSTD1 | 3491 | 2129510 | 146 | 1.65E+05 | 4.42E−12 | 2.08E−18 | 1.25E+06 |
| | pOTB7-βGUS | 3997 | 2438170 | 947 | 1.46E+04 | 3.24E−10 | 1.33E−16 | 8.01E+07 |
| Mix 2 | pENTR-PIP | 2987 | 1822070 | 232 | 1.57E+08 | 7.39E−15 | 4.06E−21 | 2.44E+03 |
| | pENTR-TACSTD1 | 3491 | 2129510 | 146 | 3.30E+05 | 2.21E−12 | 1.04E−18 | 6.26E+05 |
| | pOTB7-βGUS | 3997 | 2438170 | 947 | 1.46E+04 | 3.24E−10 | 1.33E−16 | 8.01E+07 |
| Mix 3 | pENTR-PIP | 2987 | 1822070 | 232 | 7.86E+07 | 1.48E−14 | 8.1E−21 | 4.88E+03 |
| | pENTR-TACSTD1 | 3491 | 2129510 | 146 | 6.60E+05 | 1.11E−12 | 5.19E−19 | 3.13E+05 |
| | pOTB7-βGUS | 3997 | 2438170 | 947 | 1.46E+04 | 3.24E−10 | 1.33E−16 | 8.01E+07 |
| Mix 4 | pENTR-PIP | 2987 | 1822070 | 232 | 3.93E+07 | 2.95E−14 | 1.62E−20 | 9.76E+03 |
| | pENTR-TACSTD1 | 3491 | 2129510 | 146 | 1.32E+06 | 5.53E−13 | 2.6E−19 | 1.56E+05 |
| | pOTB7-βGUS | 3997 | 2438170 | 947 | 1.46E+04 | 3.24E−10 | 1.33E−16 | 8.01E+07 |
| Mix 5 | pENTR-PIP | 2987 | 1822070 | 232 | 1.96E+07 | 5.92E−14 | 3.25E−20 | 1.96E+04 |
| | pENTR-TACSTD1 | 3491 | 2129510 | 146 | 2.64E+06 | 2.77E−13 | 1.3E−19 | 7.82E+04 |
| | pOTB7-βGUS | 3997 | 2438170 | 947 | 1.46E+04 | 3.24E−10 | 1.33E−16 | 8.01E+07 |
| Mix6 | pENTR-PIP | 2987 | 1822070 | 232 | 9.82E+06 | 1.18E−13 | 6.48E−20 | 3.90E+04 |
| | pENTR-TACSTD1 | 3491 | 2129510 | 146 | 5.28E+06 | 1.38E−13 | 6.49E−20 | 3.91E+04 |
| | pOTB7-βGUS | 3997 | 2438170 | 947 | 1.46E+04 | 3.24E−10 | 1.33E−16 | 8.01E+07 |
| Mix 7 | pENTR-PIP | 2987 | 1822070 | 232 | 4.91E+06 | 2.36E−13 | 1.3E−19 | 7.81E+04 |
| | pENTR-TACSTD1 | 3491 | 2129510 | 146 | 1.06E+07 | 6.89E−14 | 3.23E−20 | 1.95E+04 |
| | pOTB7-βGUS | 3997 | 2438170 | 947 | 1.46E+04 | 3.24E−10 | 1.33E−16 | 8.01E+07 |
| Mix 8 | pENTR-PIP | 2987 | 1822070 | 232 | 2.46E+06 | 4.72E−13 | 2.59E−19 | 1.56E+05 |
| | pENTR-TACSTD1 | 3491 | 2129510 | 146 | 2.11E+07 | 3.46E−14 | 1.62E−20 | 9.78E+03 |
| | pOTB7-βGUS | 3997 | 2438170 | 947 | 1.46E+04 | 3.24E−10 | 1.33E−16 | 8.01E+07 |
| Mix 9 | pENTR-PIP | 2987 | 1822070 | 232 | 1.23E+06 | 9.43E−13 | 5.18E−19 | 3.12E+05 |
| | pENTR-TACSTD1 | 3491 | 2129510 | 146 | 4.22E+07 | 1.73E−14 | 8.12E−21 | 4.89E+03 |
| | pOTB7-βGUS | 3997 | 2438170 | 947 | 1.46E+04 | 3.24E−10 | 1.33E−16 | 8.01E+07 |
| Mix 10 | pENTR-PIP | 2987 | 1822070 | 232 | 6.14E+05 | 1.89E−12 | 1.04E−18 | 6.24E+05 |
| | pENTR-TACSTD1 | 3491 | 2129510 | 146 | 8.45E+07 | 8.64E−15 | 4.06E−21 | 2.44E+03 |
| | pOTB7-βGUS | 3997 | 2438170 | 947 | 1.46E+04 | 3.24E−10 | 1.33E−16 | 8.01E+07 |
| Mix 11 | pENTR-PIP | 2987 | 1822070 | 232 | 3.07E+05 | 3.78E−12 | 2.07E−18 | 1.25E+06 |
| | pENTR-TACSTD1 | 3491 | 2129510 | 146 | 1.69E+08 | 4.32E−15 | 2.03E−21 | 1.22E+03 |
| | pOTB7-βGUS | 3997 | 2438170 | 947 | 1.46E+04 | 3.24E−10 | 1.33E−16 | 8.01E+07 |

Total RNA studies. One μg of total RNA isolated from MDA-MB-453 cells (Stratagene) or from human lymph nodes (Ambion) were used as templates in RT-PCR reactions conducted on the GeneXpert® in cartridges designed for RNA purification followed by RT-PCR. For each sample, the RNA was added to lysis buffer (50 mM Tris, pH 7.0, 4 M guanidine thiocyanate, 125 mM DTT) and mixed with ethanol prior to loading into the cartridge. The RNA was captured by the solid phase material and washed with 0.2M KCl, 10% PEG 8000, 20 mM Tris, pH 8.0 before eluting in 30 μL DEPC-treated water. The eluate was mixed with RT reaction components consisting of 1×PCR buffer (Invitrogen), 6 mM MgCl₂, 267 μM dNTPs, 80 nM each RT primer, 40U Roche RNAse Protector, and 2.6U Omniscript (Qiagen). An RT reaction was carried out for 5 minutes at 48° C. and subsequently inactivated at 95° C. for 30 seconds. The inactivation step was repeated a total of 2 times. To this mixture was added the PCR primers and probes to a final concentration as listed above, and 0.1 U/μL Eppendorf Taq+15 μM hot start inhibitor. Additional 10×PCR buffer was added to correct for volume changes.

The cycling protocol for MDA-MB-453 samples was a 3-stage protocol consisting of: stage 1, 95° C. for 30 seconds for 1 cycle; stage 2, 95° C. for 1 second, 60° C. for 4 seconds, and 68° C. for 6 seconds with optics “on” for 27 cycles; and Stage 3, 86° C. for 1 second, 60° C. for 4 seconds, and 68° C. for 6 seconds with optics “on” for 15 cycles. “Advance to Next Stage,” when used, was set to monitor the PIP channel and advance as soon as possible following Ct detection.

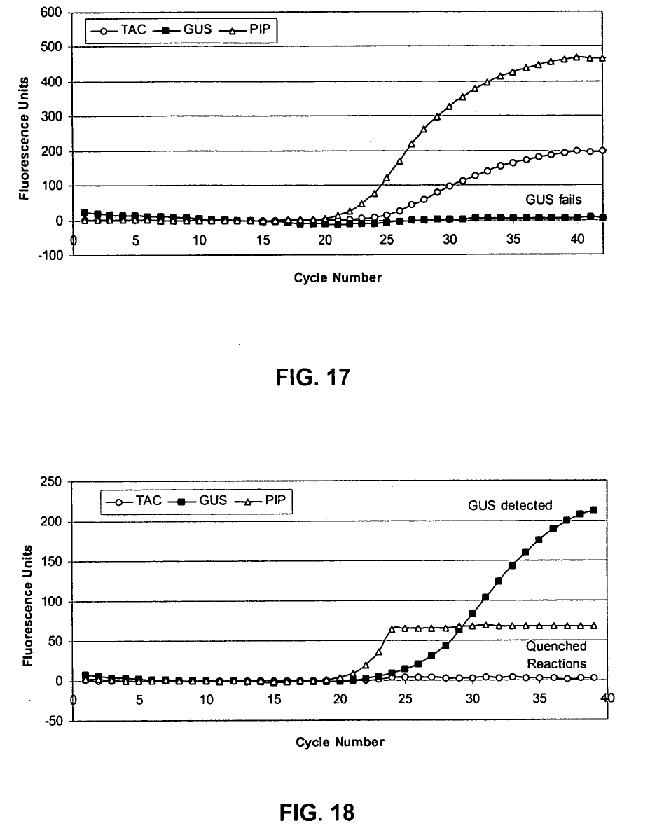

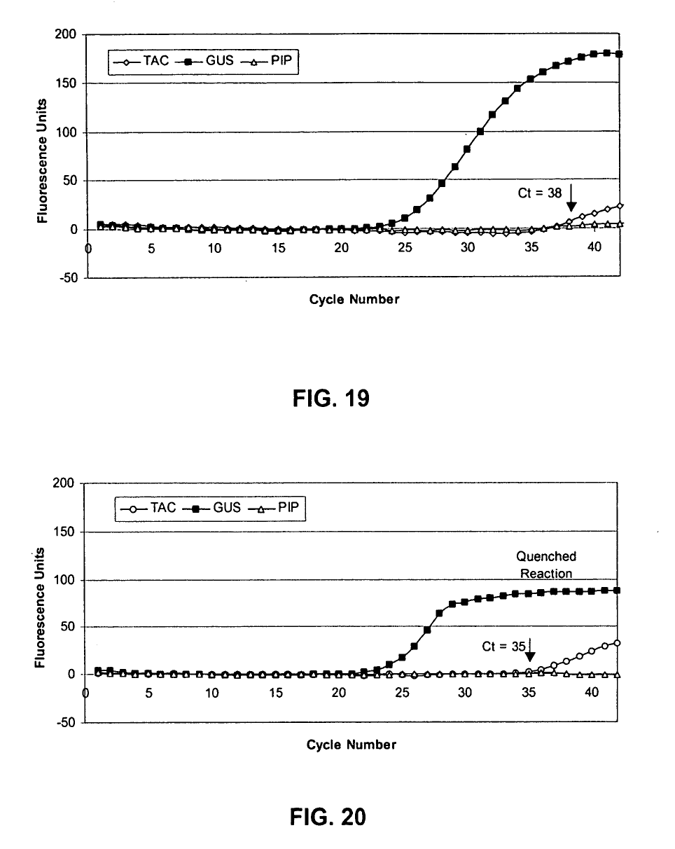

The cycling protocol for Human Lymph Node samples was a 3-stage protocol consisting of: sage 1, 95° C. for 30 seconds for 1 cycle; stage 2, 95° C. for 1 second, 60° C. for 4 seconds, and 68° C. for 6 seconds with optics “on” for 27 cycles; and stage 3, 95° C. for 1 second and 68° C. for 6 seconds with optics “on” for 15 cycles. “Advance to Next Stage,” when used, was set to monitor the β-GUS channel and advance as soon as possible following Ct detection. FIGS. 17-20 show the results of these experiments with Advance to Next Stage” turned off (FIG. 17 (MDA-MB-453 cells) and 19 (Human Lymph Node)) or turned on (FIG. 18 (MDA-MB-453 cells) and 20 (Human Lymph Node)).

These data show the effectiveness of the above-described methods of modulating relative amplification of target sequences in a triplex assay. Example 3 demonstrates the improved power of detection of metastatic breast cancer cells in sentinel lymph nodes when two targets are used in conjunction, likely due to the heterogeneous nature of cancerous tumors in the human population.

The size of individual biopsy samples is highly variable adding a layer of inherent variability into any measurements of nucleic acid detection. One approach to correct for this sample size variability is to use an endogenous control gene for internal normalization. Therefore, a 2-target assay for, for example, breast cancer metastases would require a multiplex assay design with 3 genes, each target gene plus one endogenous control gene.

As demonstrated in Example 3, for the specific example of breast cancer metastases detection in sentinel lymph nodes, there are a number of combinations of target genes that could potentially be useful in a multiplex PCR detection kit. Not all of the combinations are practicable, however. Some potential target genes are highly expressed in normal lymph node tissue and are therefore undesirable for use as a diagnostic tool. The target genes ultimately selected for inclusion in a diagnostic kit must also meet high standards of accuracy in actual clinical samples (as exemplified in the bootstrap analysis) and show tight distributions within the normal and diseased populations and a clear separation between these populations. Addition criteria include the ability to design highly cDNA-specific primers to eliminate the possibility of genomic DNA amplification, and expression ranges high enough to enable a timely delivery of assay results in an intraoperative situation. The combination that best met all these criteria is the combination of PIP and TACSTD1.

β-GUS was selected as the endogenous control gene despite its slightly lower than desirable expression level because of its extremely tight distribution in patient samples. This was considered to be the criterion of highest importance in the selection process for the endogenous control. Other criteria included ability to design highly cDNA-specific primers and high expression level. Other endogenous control genes could potentially be used in this diagnostic kit. However all possibilities that were considered that had higher expression levels than β-GUS also had broader distributions in the patient population tested.

If an endogenous control gene with very high expression level were identified that also had a tight distribution in patients, it might be possible to design a diagnostic kit in which one could a priori expect the endogenous control gene to always be more abundant than either target gene. In such a theoretical situation, the assay design could be simplified from that described in this disclosure and use more traditionally accepted methods. This might then have the potential for the design of a more robust assay. However, in the present case, no such situation was identified and no a priori knowledge could be assumed for an unknown sample. In this case, either the endogenous control gene or one of the target genes could be of highest abundance in any given sample. A dual quenching method was therefore developed to permit highly efficient and accurate PCR reactions to occur simultaneously in a multiplex assay format.

From the patient data disclosed in Example 3, the relative expression levels of the two targets genes selected use in this diagnostic kit varied over a range of approximately 5.5 Cts (˜45-fold). In Example 4, the linear dynamic range of the two targets in the presence of higher levels of endogenous control were shown to be at least 6 Cts or 64-fold. Thus no further strategy for quenching was required beyond the dual quenching strategy disclosed herein.

Having now fully described this invention, it will be understood to those of ordinary skill in the art that the same can be performed within a wide and equivalent range of conditions, formulations and other parameters without affecting the scope of the invention or any embodiment thereof.

We claim:

1. A multiplexed nucleic acid amplification reaction method comprising amplifying in a reaction mix in two or more amplification stages a first nucleic acid sequence to produce a first amplicon and a second nucleic acid to produce a second amplicon, each amplification stage comprising one or more PCR cycles, each PCR cycle comprising a denaturing step, an annealing step and an elongation step that may be conducted at the same temperature as the annealing step, the first amplicon having a first Tm and the second amplicon having a second Tm that is different from the first Tm, wherein two or more of the amplification stages have different amplicon denaturation conditions, such that relative accumulation of the first amplicon and the second amplicon is modulated.

2. The method of claim 1, comprising monitoring accumulation of the first and the second amplicons in the reaction mix.

3. The method of claim 2, wherein a first fluorescent indicator accumulates in the reaction mix as a result of the accumulation of the first amplicon and a second, different fluorescent indicator accumulates in the reaction mix as a result of the accumulation of the second amplicon.

4. The method of claim 3, wherein the amplifying is a fluorescent 5′ endonuclease assay.

5. The method of claim 3, wherein the reaction mix contains a molecular beacon or a scorpion probe specific to the first or second amplicon.

6. The method of claim 1, wherein the reaction mix contains a first primer set and a second primer set having different Tms and wherein the amplification reaction comprises two stages with different primer annealing conditions so that relative accumulation of amplicons produced by the first primer set and the second primer set is modulated between the two stages.

7. The method of claim 1 in which a third nucleic acid sequence is amplified to produce a third amplicon.

8. The method of claim 7 in which a fourth nucleic acid sequence is amplified to produce a fourth amplicon.

9. The method of claim 1, wherein the amplification reaction is conducted in thermal cycler in which cycle reaction protocols are controlled by a computer device according to the following steps:
(a) monitoring one or more reporters in a first reaction stage, wherein accumulation of the one or more reporters corresponds to accumulation of one or more amplicons in the reaction mixture; and
(b) advancing to a second reaction stage when accumulation of the one or more reporters crosses a threshold level.

10. The method of claim 9, wherein reaction protocols are changed when the reaction is advanced to a next stage.

11. The method of claim 9, further comprising conducting one or more additional cycles after threshold crossing, but before advancing to a new stage.

12. The method of claim 9, further comprising terminating the reaction if none of the monitored reporters cross the threshold.

13. The method of claim 9, further comprising terminating the reaction a fixed number of cycles after advancing to next stage.

14. The method of claim 9, wherein the threshold crossings of different reporters may advance to either the same or different stages.

15. The method of claim 9, in which the thermal cycler comprises an excitation laser and fluorescence detector configured to monitor accumulation of a fluorescent indicator in the reaction mix.

16. A method of identifying expression of markers indicative of the presence of breast cancer cells in a lymph node of a patient, comprising quantifying expression levels of TACSTD1, PIP and an endogenous control mRNA in a nucleic acid sample from a patient's lymph nodes.

17. The method of claim 16, comprising performing a multiplexed QRT-PCR reaction on the nucleic acid sample in which expression levels of TACSTD1, PIP and an endogenous control mRNA are measured.

18. The method of claim 17, in which one or more of the following primers or probes are used in the multiplexed method:
(SEQ ID NO: 5, bases 333 to 357)
CTGGGACTTTTACACCAACAGAACT;
(SEQ ID NO: 12)
GCAGATGCCTAATTCCCGAA;
(SEQ ID NO: 5, bases 386 to 405)
TGCAAATTGCAGCCGTCGTTGATGT;
(SEQ ID NO: 6, bases 348 to 368)
TCATTTGCTCAAAGCTGGCTG;
(SEQ ID NO: 13)
GGTTTTGCTCTTCTCCCAAGTTT;
(SEQ ID NO: 6, bases 371 to 402)
AAATGTTTGGTGATGAAGGCAGAAATGAATGG;
(SEQ ID NO: 15)
GCAGATGCCTAATTCCC;
(SEQ ID NO: 16)
AGCCCATCATTGTTCTG;
(SEQ ID NO: 17)
GCGGCTCCAGCTCCTGTTCAG;
(SEQ ID NO: 18)
GGCGCATTATGATCTTCCGAGTGTTGTC;
(SEQ ID NO: 5; bases 65 to 87)
CCAGCCCTGCCACCCTGCTCCTG;
(SEQ ID NO: 19)
GGGAATGTCAAAATTCTTT;
(SEQ ID NO: 20)
GCGCGTTCGGGCTTCTGCTT;
(SEQ ID NO: 21)
GCGAGTTTTCACAGACACATTCTTCCTGAG;
(SEQ ID NO: 6, bases 220 to 240)
CGCGGCGACGGCGACTTTTGC;
(SEQ ID NO: 22)
AGTTTACGGCCAGCTTG;
(SEQ ID NO: 23)
CTCATTTGGAATTTTGCCGAT;
(SEQ ID NO: 24)
CCGAGTGAAGATCCCCTT;
(SEQ ID NO: 25)
TGAACAGTCACCGACGAGAGTGCTGG;
and
(SEQ ID NO: 26)
TTTGTTGTCTCTGCCGAGT.

19. A kit comprising:
(a) packaging material;
(b) a vessel contained within the packaging material containing a first primer pair and a second primer pair, wherein in a multiplexed PCR assay, the first PCR primer pair generates an amplicon having a first Tm and the second PCR primer pair generates an amplicon having a second Tm, different from the first; and
(c) instructions describing a multiplexed nucleic acid amplification method comprising amplifying in a reaction mix a first nucleic acid sequence to produce a first amplicon and a second nucleic acid to produce a second amplicon, the first amplicon having a first Tm and the second amplicon having a second Tm that is different from the first Tm, wherein the reaction comprises two stages comprising two protocols with different amplicon denaturation conditions, such that relative accumulation of the first amplicon and the second amplicon is modulated.

20. The kit of claim 19, comprising primers for producing an amplicon specific to TACSTD1, PIP and an endogenous control mRNA.

21. The kit of claim 19, comprising one or more of the following primers or probes:
(SEQ ID NO: 5, bases 333 to 357)
CTGGGACTTTTACACCAACAGAACT;
(SEQ ID NO: 12)
GCAGATGCCTAATTCCCGAA;
(SEQ ID NO: 5, bases 386 to 405)
TGCAAATTGCAGCCGTCGTTGATGT;
(SEQ ID NO: 6, bases 348 to 368)
TCATTTGCTCAAAGCTGGCTG;
(SEQ ID NO: 13)
GGTTTTGCTCTTCTCCCAAGTTT;
(SEQ ID NO: 6, bases 371 to 402)
AAATGTTTGGTGATGAAGGCAGAAATGAATGG;
(SEQ ID NO: 15)
GCAGATGCCTAATTCCC;
(SEQ ID NO: 16)
AGCCCATCATTGTTCTG;
(SEQ ID NO: 17)
GCGGCTCCAGCTCCTGTTCAG;
(SEQ ID NO: 18)
GGCGCATTATGATCTTCCGAGTGTTGTC;
(SEQ ID NO: 5; bases 65 to 87)
CCAGCCCTGCCACCCTGCTCCTG;
(SEQ ID NO: 19)
GGGAATGTCAAAATTCTTT;
(SEQ ID NO: 20)
GCGCGTTCGGGCTTCTGCTT;
(SEQ ID NO: 21)
GCGAGTTTTCACAGACACATTCTTCCTGAG;
(SEQ ID NO: 6; bases 220 to 240)
CGCGGCGACGGCGACTTTTGC;
(SEQ ID NO: 22)
AGTTTACGGCCAGCTTG;
(SEQ ID NO: 23)
CTCATTTGGAATTTTGCCGAT;
(SEQ ID NO: 24)
CCGAGTGAAGATCCCCTT;
(SEQ ID NO: 25)
TGAACAGTCACCGACGAGAGTGCTGG;
and
(SEQ ID NO: 26)
TTTGTTGTCTCTGCCGAGT.
